(19) United States
(12) Patent Application Publication
(10) Pub. No.: US 2009/0226536 A1
(43) Pub. Date: Sep. 10, 2009

(54) METHODS AND MATERIALS RELATING TO ENHANCED PRODUCTION OF DOPAMINE NEURONS

(76) Inventors: Ernesto A. Arenas, Stockholm (SE); Goncalo Castelo Branco, Stockholm (SE); Nina Rawal, Stockholm (SE)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/574,606

(22) PCT Filed: Sep. 2, 2005
(86) PCT No.: PCT/IB05/02803

§ 371 Date: Mar. 2, 2007

Related U.S. Application Data
(60) Provisional application No. 60/606,819, filed on Sep. 2, 2004.

(51) Int. Cl. A61K 35/30; C12N 5/06; C12N 15/87; A61P 25/16; C12Q 1/02; C12Q 1/68; A61K 35/12

(52) U.S. Cl. 424/570; 435/375; 435/377; 435/455; 435/373; 435/29; 435/6; 424/93.7

(57) ABSTRACT

Induction of neuronal fate in neural stem cells or neural progenitor or precursor cells, or other stem cells, and enhancement of induction of a specific neuronal phenotype, and particularly to induction and enhancement of induction of a midbrain dopaminergic neuronal phenotype. Expressing a nuclear receptor of the Nurr1 subfamily above basal levels within the cell, and regulating GSK-3β inhibition within the cell other than by means of a ligand for a Frizzled receptor.

The present invention relates to induction of neuronal fate in neural stem cells or neural progenitor or precursor cells, or other stem cells. It relates to induction and enhancement of induction of a specific neuronal phenotype, and particularly to induction and enhancement of induction of a midbrain dopaminergic neuronal phenotype.

Aspects and embodiments of the invention involve regulation of inhibition of GSK-3β. This may involve use of a substance that is an inhibitor of GSK-3β or a substance that affects GSK-3β activity by acting on or mimicking, for example, β-catenin—e.g. to stabilise β-catenin, to inhibit its degradation or to inhibit its phosphorylation, or by acting on a substrate of GSK-3β. Reference to “inhibition of GSK-3β” or “GSK-3β inhibition” herein is reference to any of these approaches. Use of a Wnt or any ligand for a Frizzled receptor is excluded. Reference to a “GSK-3β inhibitor” is, except where context suggests otherwise, reference to use of a substance that acts directly on GSK-3β to inhibit it.

In certain embodiments, GSK-3β inhibition may be upregulated or downregulated, i.e. inhibition may be induced or reduced. Primarily herein there is reference to induction of inhibition, but analogous embodiments in which regulation of GSK-3β inhibition is by way of downregulation, i.e. reduction of inhibition, are also provided by the present invention.

Parkinson's disease (PD) is a very common neurodegenerative disorder whose pathogenesis is characterized by a selective and progressive loss of midbrain dopaminergic (DA) neurons. The enhancement of induction of neuronal phenotype has the potential to allow for treatment of Parkinson's disease and other seriously debilitating neurodegenerative disorders.

Previously, human fetal mesencephalic tissue has been grafted into Parkinsonian patients with positive results, but development of specific cell replacement therapies utilizing the present invention overcomes practical and ethical difficulties with such prior approaches. In particular, the present invention allows for development of cell preparations for transplantation while reducing or eliminating any need for use of embryo tissue or embyronic cells. Stem cells may be obtained from the umbilical cord, a tissue that is normally discarded. Another option to is to obtain adult stem cells, e.g. from bone marrow, blood, skin, eye, olfactory bulb or olfactory epithelia.

Previously (WO00/66713 and Wagner et al., 1999), the present inventors' laboratory showed that induction of dopaminergic neuronal phenotype is enhanced in cells expressing Nurr1 in the presence of one or more factors obtainable from a Type 1 astrocyte/early glial cell of the ventral mesencephalon.

Further work found that Wnt factors are useful in enhancing induction of neuronal phenotype of cells expressing Nurr1. These results were published in WO2004/029229.

In particular, as disclosed in WO2004/029229, it was found that all Wnts that are expressed in the VM at higher levels than in the dorsal midbrain by the time of birth of DA neurons are useful in inducing or promoting dopaminergic neuronal development by enhancing proliferation, self-renewal, dopaminergic induction, survival, differentiation and/or maturation in neural stem, progenitor or precursor cells, or other stem or neural cells.

It was found that:
Wnt-1 promotes the proliferation of dopaminergic precursors and the maturation of dopaminergic precursor and/or stem cells into dopaminergic neurons;
Wnt-7a promotes proliferation of dopaminergic precursors and allows their differentiation into dopaminergic neurons;
Wnt-3a promotes proliferation and/or self-renewal of dopaminergic precursor and/or stem cells;
Wnt-2 promotes cell cycle exit and the acquisition of a dopaminergic neuronal phenotype by Nurr1+ precursors; and that
Wnt-5a is the most efficient at inducing a dopaminergic phenotype in neural stem, precursor or progenitor cells, and in enhancing dopaminergic induction or differentiation in a neuronal cell.

Wnt-1 is more efficient than Wnt-3a and Wnt-5a at promoting the proliferation and maturation of dopaminergic precursor and/or stem cells.

The induction of specific neuronal phenotypes requires the integration of both genetic and epigenetic signals. In the developing midbrain, the induction of dopaminergic neurons requires the orphan nuclear receptor Nurr1 (Zetterström et al., 1997; Saucedo-Cárdenas et al., 1998; Castillo et al., 1998), but expression of Nurr1 is not sufficient to induce a dopaminergic phenotype in neural stem cells (Wagner et al., 1999). A combination of Nurr1 and an unknown soluble signal derived from developing ventral midbrain type 1 astrocytes/early glial cells is sufficient to induce a midbrain dopaminergic phenotype in neural stem cells (Wagner et al., 1999). Wnt-5a is part of such signal and members of the Wnt family of proteins, including Wnt-1, -2, -3a, -5a and -7a are developmentally regulated and differentially control the development of midbrain dopaminergic neurons. Partially purified Wnt-1, -2, -5a, and -7a, but not Wnt-3a, increased the number of E14.5 midbrain DA neurons by two different mechanisms. Wnt-1 and -7a predominantly increased the proliferation of Nurr1 precursors and allowed their differentiation into dopaminergic neurons. Wnt-2 favored cell cycle exit and the acquisition of a dopaminergic neuronal phenotype by Nurr1+ precursors. Wnt-5a mainly increased the proportion of Nurr1 precursors that acquired a neuronal DA phenotype. In agreement with these findings, Wnt-5a was as efficient as midbrain astrocytes/early glial cells at inducing dopaminergic neurons in Nurr1-expressing midbrain or cortical E13.5 precursors. Moreover, the cysteine rich domain of Frizzled 8 efficiently blocked the basal and the VM T1A-, Wnt-1 or Wnt-5a-mediated effects on the increase of cells with a dopaminergic phenotype in Nurr1-expressing neural precursor cultures, and the effect of endogenous Wnts on neural stem cells or FGF-8 expanded Nurr1+ midbrain neurospheres. Thus, the data provided indication that Wnts independently regulate, by partially different mechanisms, the generation of neurons with a DA phenotype in Nurr1-expressing precursors/stem cells.

These findings place Wnt ligands as key regulators of proliferation, self-renewal, differentiation and fate decisions during ventral midbrain neurogenesis. Moreover, Wnts may be used to guide the development of stem cells into dopaminergic neurons and thereby exploit their therapeutic potential in cell replacement therapy for Parkinson's Disease.

Embryonic, neural and multipotent stem cells have the ability to differentiate into neural cell lineages including neurons, astrocytes and oligodendrocytes. Moreover, stem cells can be isolated, expanded, and used as source material for brain transplants (Snyder, E. Y. et al. Cell 68, 33-51 (1992); Rosenthal, A. Neuron 20, 169-172 (1998); Bain et al., 1995; Gage, F. H., et al. Ann. Rev. Neurosci. 18, 159-192 (1995); Okabe et al., 1996; Weiss, S. et al. Trends Neurosci. 19, 387-393 (1996); Snyder, E. Y. et al. Clin. Neurosci. 3, 310-316 (1996); Martinez-Serrano, A. et al. Trends Neurosci. 20, 530-538 (1997); McKay, R. Science 276, 66-71 (1997); Deacon et al., 1998; Studer, L. et al. Nature Neurosci. 1, 290-295 (1998); Bjorklund and Lindvall 2000; Brustle et al., 1999; Lee et al., 2000; Shuldiner et al., 2000 and 2001; Reubinoff et al., 2000 and 2001; Tropepe et al., 2001; Zhang et al., 2001; Price and Williams 2001; Arenas 2002; Bjorklund et al., 2002; Rossi and Cattaneo, 2002; Gottlieb et al., 2002).

Most neurodegenerative diseases affect neuronal populations. Moreover, most of the damage occurs to a specific neurochemical phenotype. In human Parkinson's disease, for example, the major cell type lost is midbrain dopaminergic neurons. Functional replacement of specific neuronal populations through transplantation of neural tissue represents an attractive therapeutic strategy for treating neurodegenerative diseases (Rosenthal, A. Neuron 20, 169-172 (1998)). Another alternative would be the direct infusion of signals required to promote regeneration, repair or guide the development and/or recruitment of stem or progenitor or precursor cells, or the administration of drugs that regulate those functions.

Stem/progenitor or precursor cells are an ideal material for transplantation therapy since they can be expanded and instructed to assume a specific neuronal phenotype. These cells would circumvent ethical and practical issues surrounding the use of human fetal tissue for transplantation since tissue from several donors is currently required to treat one patient but one stem cell could be used to treat many patients.

Wnts, however, are poorly soluble. This places limitations on their use in the clinical set up and makes their application in therapeutics difficult.

The present invention relates to induction or promotion of dopaminergic neuronal phenotype in cells through inhibition of GSK-3β, e.g. by targeting GSK-3β itself or by mimicking or enhancing β-catenin activity. This provides for activation of the signalling pathways transducing Wnt signalling as a useful therapeutic approach and an alternative to Wnt ligands. Wnt ligands and any ligand of a Frizzled receptor are not used in the present invention.

GSK-3β is a multifunctional serine/threonine kinase, critical in β-catenin degradation. Ref: Doble and Woodgett J Cell Sci. 2003 Apr. 1; 116 (Pt 7):1175-86. Human accession number: NM_{—}002093.

By provision of GSK-3β inhibition in cells as disclosed herein, whether in cultures or in the brain, and whether the inhibition is direct or indirect, the invention allows the induction or promotion of: proliferation and/or self-renewal of dopaminergic precursors, progenitor or stem cells; and/or promotion of dopaminergic neuron, precursor, progenitor or stem cell survival, differentiation and maturation, increasing the yield of dopaminergic neurons; and/or induction of a neuronal dopaminergic fate in stem, progenitor, precursor or neuronal cells in vitro or in vivo.

The present invention provides for increasing neural differentiation by inhibition of GSK-3β, and further provides a method of increasing the number of dopaminergic neurons in a dopaminergic neuron population through conversion of precursors expressing nurr1 into TH+ neurons.

GSK-3β may be inhibited directly or indirectly. Inhibition indirectly may for example be by acting on upstream or downstream signaling components, e.g. by loss of function or degradation of dishevelled, axin, FRAT/GBP, or casein kinase-1, or overexpression or stabilisation or inhibition of degradation of β-catenin or inhibition of β-catenin phosphorylation. Reference to inhibition of GSK3 herein may refer to any of these approaches, indeed any approach that inhibits GSK-3β except by means of a ligand of a Frizzled receptor, e.g. Wnts, unless context indicates otherwise.

The indirubin family of GSK-3β inhibitors was initially isolated from traditional Chinese medicines used against various chronic diseases including chronic myelocytic leukaemia (19). More recently, the potential application of a GSK-3β inhibitor of the indirubin family in stem cell therapy was reported (15). Our results, describing new properties of chemical GSK-3β inhibitors on the differentiation of DA precursors, have important implications for the development of cell replacement therapies for PD. Current approaches focus on grafting human foetal midbrain DA precursors into the neostriatum of PD patients (27). The success of these approaches critically depends on the number of grafted DA neurons and more than five fetuses per patient are required (27, 28). Further potential approaches to VM precursor grafting include the transplantation of human stem cells pre-differentiated into DA neurons. However, the differentiation of human stem cells into DA neurons (29, 30) has proven to be more difficult than that reported for mouse stem cells (22, 31-33), highlighting the need for novel differentiation signals to be implemented in stem cell preparations. Our results show that GSK-3β inhibition efficiently promotes DA differentiation of precursors and provide indication such inhibition may be applied to cell preparations prior to transplantation in PD patients.

β-catenin signaling has been generally associated with proliferation of neural progenitors in the developing central nervous system (2, 34, 35). Indeed, constitutive expression of β-catenin under the control of promoters active in neural stem/progenitor cells results in an expansion of the entire neural tube (34, 35), supporting a role of β-catenin in progenitor proliferation. However, these experiments do not directly address a possible role of β-catenin in later developmental stages, during neuronal differentiation.

We have previously shown that Wnt-1 and Wnt-5a promote VM precursor proliferation and DA differentiation in VM precursor cultures, respectively. Furthermore, we found Nurr1/β-catenin co-localization and TCF/LEF transcriptional activity at E10.5 in the VM domain where DA precursors differentiate in vivo (7). These results suggested that Wnt/β-catenin signalling could regulate both proliferation and differentiation in DA precursors.

We now report that GSK-3β inhibition and β-catenin stabilization increase both the differentiation of precursors into DA neurons and the proportion of DA neurons out of the total neuronal population.

Our results are in agreement with recent publications supporting a role for Wnt/β-catenin signaling in sensory neuron differentiation (37) and dendritogenesis (10), as well as in the differentiation of other organs such as the skin (38-40). Thus, our data provide evidence for a general role of β-catenin in cell differentiation and indicate that GSK-3βinhibition might be particularly well suited for cell replacement therapies by promoting. DA differentiation in cell preparations containing both neuronal and glial precursors.

Inhibitors of GSK-3β are available in the art. GSK3 inhibitors that have been known for some time are lithium (a magnesium competitor) and Zinc. Gsk-3β is phylogenetically very closely related to CDK; research has shown that there are at least four ATP-competitive Kinase inhibitor classes that were found to act upon GSK-3β and to a lesser extent to CDKs: hymenaldisines; indirubins; aloisines; and paullones. Recently more potent and specific GSK3 inhibitors including maleimides, thiazoles derivatives, thienyl and methyl halomethyl ketones and oxidized aminopyrimidines have been identified. References include: Knockaert et al. J Biol Chem. 2002 July 12; 277(28):25493-501; Hoessel et al. (1999) Nat Cell Biol.; 1 (1): 60-67; Leclerc et al. (2001) J Biol Chem; 276 (1): 251-260; Conde et al. J. Med. Chem. 46, 4631-4633 (2003); Cline et al. Diabetes 51, 2903-2910 (2003); Ring et al. Diabetes 52, 588-595 (2003); Bhat et al. J. Biol. Chem. 278, 45937-45945 (2003); Kuo et al J. Med. Chem. 46, 4021-4031 (2003).

A variety of techniques exist in the art for identification of substances that can be used for regulation of GSK-3β inhibition. For example by assessing the capacity of the compounds being tested to inhibit the phosphorylation of GSK3 substrates (β-catenin, glycogen synthase, tau, elf2B, CREB, c-jun) but not the substrates of other enzymes (such as cyclin-dependent kinases, protein kinases-A, -B or -C, SAP kinases, JNK, MAPK, MEK, Casein kinases, Rho kinase, PDK1).

Compounds identified as being useful in GSK-3β inhibition, e.g. GSK-3β inhibitors, may be modified in order to enhance their activity by any methods known to those skilled in the art.

In preferred embodiments, inhibitors are specific for GSK-3β such as hymenaldisines, aloisines, maleimides, thiazoles, thienyl and methyl halomethyl ketones, oxidized aminopyrimidines, indirubines (e.g. Indirubin-3-monoxime) or paullones (kenpaullone). In a particularly preferred embodiment, the inhibitor is kenpaullone. References: Leclerc et al. (2001) J Biol Chem; 276 (1): 251-260; Hoessel et al. (1999) Nat Cell Biol.; 1 (1): 60-67; Polychronopoulos et al. J Med. Chem. 2004 Feb. 12; 47(4):935-46; Conde et al. J. Med. Chem. 46, 4631-4633 (2003); Cline et al. Diabetes 51, 2903-2910 (2003); Ring et al. Diabetes 52, 588-595 (2003); Bhat et al. J. Biol. Chem. 278, 45937-45945 (2003); Kuo et al. J. Med. Chem. 46, 4021-4031 (2003).

Any aspect or embodiment of the invention can apply to or use a neuronal cell i.e. a neuron. A ‘neural cell’ in the present disclosure may be a neuronal cell.

Cell preparations rich in dopaminergic neurons may be used for cell replacement therapy in Parkinson's disease or other disorders, and for studying signalling events in dopaminergic neurons and the effects of drugs on dopaminergic neurons in vitro, for instance in high throughput screening.

Aspects and embodiments of the present invention are provided as set out in the claims below.

In one aspect, the present invention provides a method of inducing a dopaminergic neuronal fate in a stem cell, neural stem cell or neural progenitor or precursor cell, or enhancing dopaminergic induction or differentiation in a neuronal cell, or expanding a dopaminergic precursor or progenitor or a Nurr1-expressing stem cell, the method comprising: regulating GSK-3β inhibition (e.g. by treating the cell with an inhibitor of GSK-3β, overexpression or providing β-catenin, inhibiting β-catenin phosphorylation or inhibiting β-catenin degradation, or other regulation of GSK-3β inhibition) whereby dopaminergic neurons are produced, wherein the method optionally comprises previously expressing a nuclear receptor of the Nurr1 subfamily above basal levels within the cell.

Regulating, e.g. inducing, GSK-3β inhibition (or indirectly inhibiting GSK-3β as discussed) may be in vivo, ex vivo, in vitro or in culture. Treatment in vitro, ex vivo or in culture may be preferred.

In methods of the invention, treating with a substance that acts in GSK-3β inhibition may be by means of contacting a cell with the substance. Treating with such a substance, e.g. a GSK-3β inhibitor, may be by means of provision of the substance to a culture comprising the stem, progenitor or precursor cell, or to such a cell in vivo. If instead of treating with a GSK-3β inhibitor the inhibition of GSK-3β is to be indirect, then the treatment that causes the indirect inhibition, e.g. by stabilising β-catenin, causing overexpression of β-catenin and so on, may similarly be performed in vivo, or, possibly preferred, in vitro, ex vivo or in culture.

In addition to provision of a substance that acts in GSK-3β inhibition, the stem cell or neural stem, progenitor or precursor cell or neuronal cell may be in co-culture with Type 1 astrocytes/glial cells, or in contact with such cells or factors derived from them in vitro or in vivo.

The co-cultured or host cell may be another stem, neural stem, progenitor, precursor or neural cell.

Nurr1 (Law, et al., 1992; Xing, et al., 1997; Castillo, 1997; GenBank nos. S53744, U72345, U86783) is a transcription factor of the thyroid hormone/retinoic acid nuclear receptor superfamily. As shown previously in WO00/66713 and Wagner et al., 1999, expression of Nurr1 above basal levels in neural stem cells or neural progenitor cells increases the proportion of the cells which differentiate toward a neuronal fate. The induction of a neuronal fate may be carried out in vitro or in vivo. The ability to induce differentiation of stem cells or neural stem, progenitor or precursor cells toward the neuronal fate prior to, or following transplantation, could also be enhanced by glial-derived signals as previously shown in WO00/66713 and Wagner et al., 1999.

Nurr1 is a member of the NR4A subfamily. Methods of the invention are not limited to use of Nurr1, although Nurr1 may be preferred, and methods may comprise expressing any nuclear receptor of the NR4A subfamily above basal levels in the cell. Receptors of the NR4A subfamily include Nurr1/NR4A2, Norl/NR4A3 and NGFI-B/NR4A1. Thus, in methods of the invention, a nuclear receptor of the NR4A subfamily (e.g. Nurr1, Nor1 or NGFI-B) may be expressed above basal levels within the cell. Accession numbers for example NR4A subfamily members are as follows:

NGF-IB protein: NP775181 NP775180 NP002126;
NGFI-B nucleotide: NM_{—}173158 NM_{—}173157 NM_{—}002135;
Nor-1 protein: NP775292 NP775291 NP775290 NP008912S71930 Q92570;
Nor-1 nucleotide: NM_{—}005413 NM_{—}173200 NM_{—}173199 NM_{—}173198 NM_{—}006981.

Members of the Wnt family of glycoproteins are poorly soluble (Bradley and Brown, 1990 and 1995) and expressed in the developing mesencephalon (Parr et al., 1993). Wnts regulate midbrain-hindbrain development (McMahon and Bradley, 1990; Thomas and Capecchi, 1990), neural patterning (Kiecker and Niehrs, 2001; Nordstrom et al., 2002; Houart et al., 2002), precursor proliferation (Taipale and Beachy, 2001; Chem and Walsh, 2002; Megason and McMahon, 2002) and fate decisions in multiple tissues (Kispert et al., 1998; Ross et al., 2000; Hartmann and Tabin, 2001; Marvin et al., 2001; Schneider and Mercola, 2001; Tzahor and Lassar, 2001; Pandur et al., 2002), including the nervous system (Dorsky et al., 1998; Baker et al., 1999; Wilson et al., 2001; Garcia-Castro et al., 2002; Muroyama et al., 2002).

As used herein, a “Wnt polypeptide”, “Wnt glycoprotein” or “Wnt ligand” refers to a member of the Wingless-int family of secreted proteins that regulate cell-to-cell interactions. Wnts are highly conserved from Drosophila and Caenorhabditis elegans, to Xenopus, zebra fish and mammals. The 19 Wnt proteins currently known in mammals bind to two cell surface receptor types: the seven transmembrane domain Frizzled receptor family, currently formed by 10 receptors, and the Low density lipoprotein-receptor related proteins (LRP) 5 and 6 and the kremen 1 and 2 receptors. The signal conveyed by Wnts is transduced via three known signalling pathways: (1) the so called canonical signalling pathway, in which GSK-3β is inhibited, does not phosphorylate β-catenin, which is then not degraded and is translocated to the nucleus to form a complex with TCF and activate transcription of Wnt target genes; (2) the planar polarity and convergence-extension pathway, via Jnk; And (3) the inositol 1,4,5 triphosphate (IP3)/calcium pathway, in which calcineurin dephosphorylates and activates the nuclear factor of activated T cells (NF-AT) (Saneyoshi et al. Nature. 2002 May 16; 417(6886):295-9). For review see the Wnt home page, findable on the web using any available browser (www.stanford.edu/˜rnusse/wntwindow.html). Other co-receptors involved in Wnt signaling include the tyrosine kinase receptor Rorl and Ror 2 (Oishi I et al. Genes Cells. 2003 July; 8(7):645-54), the derailed/RYK receptor family (Yoshikawa et al., Nature. 2003 Apr. 10; 422(6932):583-8) which encode catalytically inactive receptor tyrosine kinases.

By “stem cell” is meant any cell type that can self renew and, if it is an embryonic stem (ES) cell, can give rise to all cells in an individual, or, if it is a multipotent or neural stem cell, can give rise to all cell types in the nervous system, including neurons, astrocytes and oligodendrocytes. A stem cell may express one or more of the following markers: Oct-4, Sox1-3, Nanog (Chambers et al., 2003, Cell, May 30; 113(5):643-55., stage specific embryonic antigens (SSEA-1, -3, and -4), and the tumour rejection antigens TRA-1-60 and -1-81, as described (Tropepe et al. Neuron. 2001 April; 30(1):65-78; Xu et al. Nat. Biotechnol. 2001 October; 19(10):971-4). A neural stem cell may express one or more of the following markers: Nestin; the p75 neurotrophin receptor; Notchl, SSEA-1 (Capela and Temple Neuron. 2002 Aug. 29; 35(5):865-75).

By “neural progenitor cell” is meant a daughter or descendant of a neural stem cell, with a more differentiated phenotype and/or a more reduced differentiation potential compared to the stem cell. By precursor cell it is meant any other cell being or not in a direct lineage relation with neurons during development but that under defined environmental conditions can be induced to transdifferentiate or redifferentiate or acquire a neuronal phenotype.

A stem cell, neural stem cell or neural progenitor or precursor cell may be obtained or derived from any embryonic, foetal or adult tissue, including bone marrow, skin, eye, nasal epithelia, or umbilical cord, or region of the nervous system, e.g. from the cerebellum, the ventricular zone, the sub-ventricular zone, the striatum, the midbrain, the hindbrain, the cerebral cortex or the hippocampus. It may be obtained or derived from a vertebrate organism, e.g. from a mammal, which may be human or non-human, such as rabbit, guinea pig, rat, mouse or other rodent, cat, dog, pig, sheep, goat, cattle, horse, or primate, or from a bird, such as a chicken.

In preferred embodiments of the present invention, adult stem/progenitor/precursor cells are used, in vitro, ex vivo or in vivo. This requires a consenting adult (e.g. from which the cells are obtained) and approval by the appropriate ethical committee. If a human embryo/foetus is used as a source, the human embryo is one that would otherwise be destroyed without use, or stored indefinitely, especially a human embryo created for the purpose of IVF treatment for a couple having difficulty conceiving. IVF generally involves creation of human embryos in a number greater than the number used for implantation and ultimately pregnancy. Such spare embryos may commonly be destroyed. With appropriate consent from the people concerned, in particular the relevant egg donor and/or sperm donor, an embryo that would otherwise be destroyed can be used in an ethically positive way to the benefit of sufferers of severe neurodegenerative disorders such as Parkinson's disease. The present invention itself does not concern the use of a human embryo in any stage of its development. As noted, the present invention minimizes the possible need to employ a material derived directly from a human embryo, whilst allowing for development of valuable therapies for terrible diseases.

In some preferred embodiments, a stem or progenitor or precursor cell is contacted with a substance that acts in GSK-3β inhibition, such as a GSK-3β inhibitor or other means for inhibiting GSK-3β as discussed, and otherwise treated and/or used in accordance with any aspect of the present invention is obtained from a consenting adult or child for which appropriate consent is given, e.g. a patient with a disorder that is subsequently treated by transplantation back into the patient of neurons generated in accordance with the invention, and/or treated with GSK-3β inhibition, a GSK-3β inhibitor or a substance that inhibits GSK-3β indirectly, and/or one or more type 1 astrocyte/early glial cell-derived factors to promote or induce endogenous dopaminergic neuron development or function.

The neuronal fate to which the stem or progenitor or precursor cell is induced may exhibit an undifferentiated phenotype or a primitive neuronal phenotype. It may be a totipotent cell, capable of giving rise to any cell type in an individual, or a multipotent cell which is capable of giving rise to a plurality of distinct neuronal phenotypes, or a precursor or progenitor cell, capable of giving rise to more limited phenotype during normal development but capable of giving rise to other cells when exposed to appropriate environmental factors in vitro. It may lack markers associated with specific neuronal fates, e.g. tyrosine hydroxylase.

In a method of inducing a neuronal fate according to the present invention wherein VM precursors are subject to regulation of GSK-3β inhibition, especially induction of GSK-3β inhibition, e.g. by means of a GSK-3β inhibitor or otherwise subject to inhibition of GSK-3β, an increased proportion of cells may be induced to adopt a neuronal fate. Dopaminergic induction or differentiation may be enhanced in neuronal cells. In preferred embodiments, there is a three to five-fold increase in the proportion of TH+/TuJ1+ cells in the ventral mesencephalon.

Culture conditions could include but would not be limited to culturing the cells at different densities, in different media, on different substrates or in the presence of other cell populations.

By “expressing Nurr1 above basal levels within the cell” is meant expressing Nurr1 at levels greater than that at which it is expressed in the (unmodified) cell in vivo under non-pathological conditions. Likewise, expressing a nuclear receptor of the Nurr1 subfamily above basal levels within the cell means expressing the nuclear receptor at levels greater than that at which it is expressed in the (unmodified) cell in vivo under non-pathological conditions. Expression above basal levels includes transcriptional, translational, posttranslational, pharmacological, artificial upregulation and over-expression. Expression of nuclear receptors above basal levels is described herein with reference to Nurr1. This disclosure is also applicable to other members of the Nurr1 subfamily and may be used in methods of the invention with other nuclear receptors of that subfamily e.g. Norl or NGFI-B. Thus, although Nurr1 is exemplified, methods of the invention are not limited to Nurr1 and extend to any nuclear receptor of the Nurr1 subfamily.

Expression of Nurr1 above basal levels may be achieved by any method known to those skilled in the art. By way of example, expression above basal levels may be induced by regulating the regulation of native genomic Nurr1. This may be done by inhibiting or preventing degradation of Nurr1 mRNA or protein or by increasing transcription and/or translation of Nurr1, e.g. by contacting the cell with fibroblast growth factor 8 (FGFB), which upregulates transcription of Nurr1 (Rosenthal, A., (1998) Cell, 93 (5),755-766), and/or by introducing heterologous regulatory sequences into or adjacent the native regulatory region of Nurr1, and/or by replacing the native regulatory region of Nurr1 with such heterologous regulatory sequences, e.g. by homologous recombination, and/or by disrupting or downregulating molecules that negatively regulate, block or downregulate transcription, translation or the function of Nurr1, e.g. Nurr2 (Ohkura, et al., (1999) Biochim Biophys Acta 14444: 69-79), and/or by microinjection of Nurr1 mRNA or protein directly into the cells.

Transcription may be increased by providing the stem, neural stem, precursor, progenitor or neural cell with increased levels of a transcriptional activator, e.g. by contacting the cell with such an activator or by transformation of the cell with nucleic acid encoding the activator. Alternatively, transcription may be increased by transforming the cell with antisense nucleic acid to a transcriptional inhibitor of Nurr1.

Accordingly, a method of the present invention of inducing or enhancing induction of a neuronal fate in a stem, neural stem, precursor, progenitor cell, or neural cell, may include contacting the cell with FGF8 or FGF20 (Ohmachi et al., 2000).

As an alternative or addition to increasing transcription and/or translation of endogenous Nurr1, expression of Nurr1 above basal levels may be caused by introduction of one or more extra copies of Nurr1 into the stem, neural stem, precursor, progenitor or neural cell.

Accordingly, in a further aspect, the present invention provides a method of inducing a neuronal fate and/or enhancing the induction of dopaminergic development in a stem cell, neural stem cell, neural progenitor, precursor or neural cell, or enhancing dopaminergic induction or differentiation in a neuronal cell, the method including, in addition to contacting the cell with a substance that regulates GSK-3β inhibition, e.g. GSK-3β inhibitor or means for inhibiting GSK-3β, transforming the cell with Nurr1.

Transformation of the stem, neural stem, precursor or progenitor cell or neuronal cell may be carried out in vitro, in vivo or ex vivo. The neuronal fate to which the cell is induced may be of the type discussed herein, e.g. it may exhibit a primitive neuronal phenotype and may lack markers associated with specific neuronal fates. The invention further provides a stem cell, neural stem cell or neural progenitor or precursor cell transformed with Nurr1 and contacted with GSK-3β inhibition.

Transformed Nurr1 may be contained on an extra-genomic vector or it may be incorporated, preferably stably, into the genome. It may be operably-linked to a promoter which drives its expression above basal levels in stem cells, or neural stem, precursor or progenitor cells, or neuronal cells, as is discussed in more detail below.

“Operably linked” means joined as part of the same nucleic acid molecule, suitably positioned and oriented for transcription to be initiated from the promoter.

Methods of introducing genes into cells are well known to those skilled in the art. Vectors may be used to introduce Nurr1 into stem, or neural stem, precursor or progenitor cells or neuronal cells, whether or not the Nurr1 remains on the vector or is incorporated into the genome. Suitable vectors can be chosen or constructed, containing appropriate regulatory sequences, including promoter sequences, terminator fragments, polyadenylation sequences, enhancer sequences. Vectors may contain marker genes and other sequences as appropriate. The regulatory sequences may drive expression of Nurr1 and/or inhibition of GSK-3β (whether direct or indirect) within the stem, or neural stem, precursor or progenitor cells or neural cells. For example, the vector may be an extra-genomic expression vector, or the regulatory sequences may be incorporated into the genome with Nurr1. Vectors may be plasmids or viral.

Nurr1 may be placed under the control of an externally inducible gene promoter to place it under the control of the user. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Whatever the level of expression is in the absence of the stimulus, expression from any inducible promoter is increased in the presence of the correct stimulus. An example of an inducible promoter is the Tetracyclin ON/OFF system (Gossen, et al., 1995) in which gene expression is regulated by tetracyclin analogs.

For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992 or later edition.

Marker genes such as antibiotic resistance or sensitivity genes may be used in identifying clones containing nucleic acid of interest, as is well known in the art. Clones may also be identified or further investigated by binding studies, e.g. by Southern blot hybridisation.

Nucleic acid including Nurr1 may be integrated into the genome of the host stem, neural stem, progenitor, precursor or neural cell. Integration may be promoted by including in the transformed nucleic acid sequences which promote recombination with the genome, in accordance with standard techniques. The integrated nucleic acid may include regulatory sequences able to drive expression of the Nurr1 gene in a stem cell, or neural stem, progenitor or precursor cells, or neuronal cells.

The nucleic acid may include sequences which direct its integration to a site in the genome where the Nurr1 coding sequence will fall under the control of regulatory elements able to drive and/or control its expression within the stem, or neural stem, precursor or progenitor cell, or neuronal cell. The integrated nucleic acid may be derived from a vector used to transform Nurr1 into the stem cell, or neural stem, precursor or progenitor cells, or neuronal cells, as discussed herein.

The introduction of nucleic acid comprising Nurr1, whether that nucleic acid is linear, branched or circular, may be generally referred to without limitation as “transformation”. It may employ any available technique. Suitable techniques may include calcium phosphate transfection, DEAE-Dextran, PEI, electroporation, mechanical techniques such as microinjection, direct DNA uptake, receptor mediated DNA transfer, transduction using retrovirus or other virus and liposome- or lipid-mediated transfection. When introducing a chosen gene construct into a cell, certain considerations must be taken into account, well known to those skilled in the art. It will be apparent to the skilled person that the particular choice of method of transformation to introduce Nurr1 into a stem cell, or neural stem, precursor or progenitor cells or a neuronal cell is not essential to or a limitation of the invention.

Suitable vectors and techniques for in vivo transformation of stem cells, or neural stem, precursor or progenitor cells or neuronal cells with Nurr1 are well known to those skilled in the art. Suitable vectors include adenovirus, adeno-associated virus papovavirus, vaccinia virus, herpes virus, lentiviruses and retroviruses. Disabled virus vectors may be produced in helper cell lines in which genes required for production of infectious viral particles are expressed. Suitable helper cell lines are well known to those skilled in the art. By way of example, see: Fallaux, F. J., et al., (1996) Hum Gene Ther 7(2), 215-222; Willenbrink, W., et al., (1994) J Virol 68(12), 8413-8417; Cosset, F. L., et al., (1993) Virology 193(1), 385-395; Highkin, M. K., et al., (1991) Poult Sci 70(4), 970-981; Dougherty, J. P., et al., (1989) J Virol 63(7), 3209-3212; Salmons, B., et al., (1989) Biochem Biophys Res Commun 159(3), 1191-1198; Sorge, J., et al., (1984) Mol Cell Biol 4(9), 1730-1737; Wang, S., et al., (1997) Gene Ther 4(11), 1132-1141; Moore, K. W., et al., (1990) Science 248(4960), 1230-1234; Reiss, C. S., et al., (1987) J Immunol 139(3), 711-714. Helper cell lines are generally missing a sequence which is recognised by the mechanism which packages the viral genome. They produce virions which contain no nucleic acid. A viral vector which contains an intact packaging signal along with the gene or other sequence to be delivered (e.g. Nurr1) is packaged in the helper cells into infectious virion particles, which may then be used for gene delivery to stem cells, or neural stem, precursor or progenitor cells or neuronal cells.

As an alternative or addition to increasing transcription and/or translation of endogenous Nurr1, expression of Nurr1 above basal levels may be caused by introduction of one or more extra copies of Nurr1 protein into the stem, neural stem, precursor, progenitor or neural cell by microinjection or other carrier-based or protein delivery system including cell penetrating peptides, i.e.: TAT, transportan, Antennapedia penetratin peptides (Lindsay 2002).

In a further aspect, the present invention provides a method of inducing a specific neuronal fate in a stem, neural stem or progenitor or precursor cell, or neuronal cell, wherein the stem cell or progenitor cell or neuronal cell expresses Nurr1 above basal levels, the method including contacting the cells with a substance that regulates inhibition of GSK-3β, e.g. an inhibitor of GSK-3β or substance causing overexpression or stabilisation or inhibition of degradation of β-catenin or inhibition of β-catenin phosphorylation or other GSK-3β inhibition inducing substance as discussed, and optionally one or more factors supplied by or derived from a Type 1 astrocyte/glial cell. The factor or factors may be provided by co-culturing or contacting the stem, progenitor or precursor cell or neuronal cell with a Type 1 astrocyte/glial cell. The method may occur in vitro or in vivo. The stem cell or neural stem, precursor or progenitor cells or neuronal cells expressing Nurr1 above basal levels may be produced by transformation of the cells with Nurr1.

The factor or factors may be supplied by or derived from an immortalized astrocyte/glial cell. The factor or factors may be supplied by or derived from a glial cell line, e.g. an astrocyte or radial glia or immature glial mesencephalic cell line. Cell lines provide a homogenous cell population.

Important aspects of the present invention are based on the finding that, whereas dopaminergic neurons can be generated from stem cells or progenitor or precursor cells in vitro by a process including expression of Nurr1 above basal levels in the cells and contact of the cells with one or more factors supplied by or derived from Type 1 astrocytes/early glial cells of the ventral mesencephalon, induction of dopaminergic fate is enhanced or promoted by contact with the GSK-3β inhibitory substance.

The present invention allows for generation of large numbers of dopaminergic neurons. These dopaminergic neurons may be used as source material to replace cells which degenerate or are damaged or lost in Parkinson's disease.

The cell expressing Nurr1 above basal levels may be mitotic when it is contacted with the means for inhibiting GSK-3β.

In methods of the invention, the cell may additionally be contacted with one or more agents selected from: basic fibroblast growth factor (bFGF); epidermal growth factor (EGF); and an activator of the retinoid X receptor (RXR), e.g. the synthetic retinoid analog SR11237, (Gendimenico, G. J., et al., (1994) J Invest Dermatol 102(5), 676-80), 9-cis retinol or docosahexanoic acid (DHA) or LG849 (Mata de Urquiza et al., 2000). Treating cells in accordance with the invention with one or more of these agents may be used to increase the proportion of the stem, progenitor or precursor cells which adopt a dopaminergic fate, or enhance dopaminergic induction or differentiation in a neuronal cell, as demonstrated experimentally below. The method of inducing a dopaminergic fate or enhancing dopaminergic induction or differentiation in a neuronal cell in accordance with the present invention may include contacting the cell with a member of the FGF family of growth factors, e.g. FGF4, FGF8 or FGF20.

Advantageously, the cells may be contacted with two or more of the above agents. The inventors have unexpectedly found that the beneficial effects of bFGF or EGF and SR11237 are additive at saturating doses. This finding suggests that these agents may act through different mechanisms.

The method of inducing a dopaminergic phenotype may include pretreating the stem cell, neural stem cell or neural progenitor or precursor cell or neuronal cell with bFGF and/or EGF prior to contacting it with means for regulating GSK-3β inhibition or inhibiting GSK-3β (whether a direct GSK-3β inhibitor or means for indirectly inhibiting GSK-3β, e.g. by overexpression or stabilisation or inhibition of degradation of β-catenin or inhibition of β-catenin phosphorylation) and optionally one or more further factors supplied by or derived from Type 1 astrocytes/glial cells of the ventral mesencephalon, e.g. prior to contacting or co-culturing it with ventral mesencephalic Type 1 astrocytes/glial cells or factors derived from them.

The optional pretreatment step arises from two further unexpected findings of the inventors that were previously reported in WO60/66713 and Wagner et al. (1999): (i) that neural stem cell lines expressing Nurr1 above basal levels and showing high proliferation demonstrate enhanced induction to dopaminergic fate when co-cultured with Type 1 astrocytes/glial cells of the ventral mesencephalon; and (ii) that after treatment with bFGF or EGF in serum-free medium (SFM), the baseline proliferation of most stem cell lines expressing Nurr1 above basal levels remained elevated after passage into SFM alone.

The method of inducing a dopaminergic phenotype may include pretreating a stem cell or neural stem, progenitor or precursor cell with a member of the FGF family of growth factors, e.g. FGF2, FGF4, FGF8 or FGF20.

The cells may additionally be treated with one or more Wnt ligands, e.g. as disclosed in WO2004/029229.

A method according to the invention in which a neuronal fate is induced in a stem, neural stem or progenitor or precursor cell or there is enhanced dopaminergic induction or differentiation in a neuronal cell, may include detecting a marker for the neuronal fate. β-tubulin III (TuJ1) is one marker of the neuronal fate (Menezes, J. R., et al., (1994) J Neurosci 14(9), 5399-5416). Other neuronal markers include neurofilament and MAP2. If a particular neuronal phenotype is induced, the marker should be specific for that phenotype. For the dopaminergic fate, expression of tyrosine hydroxylase (TH), dopamine transporter (DAT) and dopamine receptors may be detected e.g. by immunoreactivity or in situ hybridization. Tyrosine hydroxylase is a major marker for DA cells. Contents and/or release of dopamine and metabolites may be detected e.g. by High Pressure Liquid Chromatography (HPLC) (Cooper, J. R., et al., The Biochemical Basis of Neuropharmacology, 7th Edition, (1996) Oxford University Press). The absence of Dopamine β hydroxylase and GABA or GAD (in the presence of TH/dopamine/DAT) is also indicative of dopaminergic fate. Additional markers include Aldehyde dehydrogenase type 2 (ADH-2), GIRK2, Lmx1b and Ptx3.

Detection of a marker may be carried out according to any method known to those skilled in the art. The detection method may employ a specific binding member capable of binding to a nucleic acid sequence encoding the marker, the specific binding member comprising a nucleic acid probe hybridisable with the sequence, or an immunoglobulin/antibody domain with specificity for the nucleic acid sequence or the polypeptide encoded by it, the specific binding member being labelled so that binding of the specific binding member to the sequence or polypeptide is detectable. A “specific binding member” has a particular specificity for the marker and in normal conditions binds to the marker in preference to other species. Alternatively, where the marker is a specific mRNA, it may be detected by binding to specific oligonucleotide primers and amplification in e.g. the polymerase chain reaction.

Nucleic acid probes and primers may hybridize with the marker under stringent conditions. Suitable conditions include, e.g. for detection of marker sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of marker sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

In a further aspect, the present invention provides a neuron produced in accordance with any one of the methods disclosed herein. The neuron may have a primitive neuronal phenotype. It may be capable of giving rise to a plurality of distinct neuronal phenotypes. The neuron may have a particular neuronal phenotype, the phenotype being influenced by the type of astrocytes/glial cells from which the factor or factors which contacted the stem, neural stem, progenitor, precursor or neural cell expressing Nurr1 above basal levels were supplied or derived, and/or by the type of astrocyte/glial cell with which the stem, neural stem, progenitor, precursor or neural cell was co-cultured or contacted. In preferred embodiments, the neuron has a dopaminergic phenotype.

The neuron may contain nucleic acid encoding a molecule with neuroprotective or neuroregenerative properties operably linked to a promoter which is capable of driving expression of the molecule in the neuron. The promoter may be an inducible promoter, e.g. the TetON chimeric promoter, so that any damaging over-expression may be prevented. The promoter may be associated with a specific neuronal phenotype, e.g. the TH promoter or the Nurr1 promoter.

The encoded molecule may be such that its expression renders the neuron independent of its environment, i.e. such that its survival is not dependent on the presence of one or more factors or conditions in e.g. the neural environment into which it is to be implanted. By way of example, the neuron may contain nucleic acid encoding one or more of the neuroprotective or neuroregenerative molecules described below operably linked to a promoter which is capable of driving expression of the molecule in the neuron.

In addition or alternatively, expression of the encoded molecule may function in neuroprotection or neuroregeneration of the cellular environment surrounding that neuron. In this way, the neuron may be used in a combined cell and gene therapy approach to deliver molecules with neuroprotective and neuroregenerative properties.

Examples of molecules with neuroprotective and neuroregenerative properties include:

(i) neurotropic factors able to compensate for and prevent neurodegeneration. One example is glial derived neurotropic growth factor (GDNF) which is a potent neural survival factor, promotes sprouting from dopaminergic neurons and increases tyrosine hydroxylase expression (Tomac, et al., (1995) Nature, 373, 335-339; Arenas, et al., (1995) Neuron, 15, 1465-1473). By enhancing axonal elongation GDNF, GDNF may increase the ability of the neurons to inervate their local environment. Other neurotropic molecules of the GDNF family include Neurturin, Persephin and Artemin. Neurotropic molecules of the neurotropin family include nerve growth factor (NGF), brain derived neurotropic factor (BDNF), and neurotropin-3, -4/5 and -6. Other factors with neurotrophic activity include members of the FGF family for instance FGF2, 4, 8 and 20; members of the Wnt family, including Wnt-1, -2, -5a, -3a and 7a; members of the BMP family, including BMP2, 4, 5 and 7, nodal, activins and GDF; and members of the TGFalpha/beta family.

(ii) antiapoptotic molecules. Bcl2 which plays a central role in cell death. Over-expression of Bcl2 protects neurons from naturally occurring cell death and ischemia (Martinou, et al., (1994) Neuron, 1017-1030). Another antiapoptotic molecule specific for neurons is BclX-L.

(iii) axon regenerating and/or elongating and/or guiding molecules which assist the neuron in innervating and forming connections with its environment, e.g. ephrins. Ephrins define a class of membrane-bound ligands capable of activating tyrosine kinase receptors. Ephrins have been implicated in neural development (Irving, et al., (1996) Dev. Biol., 173, 26-38; Krull, et al., (1997) Curr. Biol. 7, 571-580; Frisen, et al., (1998) Neuron, 20, 235-243; Gao, et al., (1996) PNAS, 93, 11161-11166; Torres, et al., (1998) Neuron, 21, 1453-1463; Winslow, et al., (1995) Neuron, 14, 973-981; Yue, et al., (1999) J Neurosci 19(6), 2090-2101. Other families of molecules involved in axon-guidance include netrins, semaphorins and slits (Nakamoto et al. Curr Biol. 2004 Feb. 3; 14 (3):R121-3).

(iv) transcription factors, e.g. the homeobox domain protein Ptx3 (Smidt, M. P., et al., (1997) Proc Natl Acad Sci USA, 94(24), 13305-13310), Lmx1b, Pax2, Pax5, Pax8, or engrailed 1 or 2 (Wurst and Bally-Cuif, 2001; Rhinn and Brand, 2001), or neurogenic genes of the basic helix-loop-helix family.

A neuron in accordance with or for use in the present invention may be substantially free from one or more other cell types, e.g. from stem, neural stem, precursor or progenitor cells. Neurons may be separated from neural stem or progenitor cells using any technique known to those skilled in the art, including those based on the recognition of extracellular epitopes by antibodies and magnetic beads or fluorescence activated cell sorting (FACS). By way of example, antibodies against extracellular regions of molecules found on stem, neural stem, precursor or progenitor cells but not on neurons may be employed. Such molecules include Notch 1, CD133, SSEA1, prominin1/2, RPTPb/phosphocan, TIS21 and the glial cell line derived neurotrophic factor receptors GFR alphas or NCAM. Stem cells bound to antibodies may be lysed by exposure to complement, or separated by, e.g. magnetic sorting (Johansson, et al., (1999) Cell, 96, 25-34). If antibodies which are xenogeneic to the intended recipient of the neurons are used, then any e.g. stem, neural stem or progenitor or precursor cells which escape such a cell sorting procedure are labelled with xenogeneic antibodies and are prime targets for the recipient's immune system. Alternatively, cells that acquire the desired phenotype could also be separated by antibodies against extracellular epitopes or by the expression of transgenes including fluorescent proteins under the control of a cell type specific promoter. By way of example dopaminergic neurons could be isolated with fluorescent proteins expressed under the control of TH, DAT, Ptx3, Nurr1or other promoters specifically used by dopaminergic neurons.

Methods of the invention may comprise additional negative or positive selection methods to enrich for neural stem, progenitor or precursor cells, or other stem or neural cells with the desired phenotype.

Negative selection may be used to enrich for DA neurons. Selective neurotoxins for non-DA neurons may be used, for instance 5-7-dihydroxytryptamine (to eliminate serotoninergic neurons), or antibodies coupled to saponin or a toxin or after addition of complement, for instance antibodies against GABA or serotonin transporter (to eliminate GABAergic or serotoninergic neurons). Methods of the invention may comprise additionally treating or contacting a neural stem, progenitor or precursor cell, or other stem or neural cell with a negative selection agent, preferably in vitro, e.g. by adding the negative selection agent to an in vitro culture containing the cell, or by culturing the cell in the presence of the negative selection agent. A negative selection agent selects against cell types other than the desired cell type(s). For example, where the invention relates to promoting, enhancing or inducing a dopaminergic neuronal phenotype, the negative selection agent may select against cells other than DA neurons and cells that develop into DA neurons such as stem cells and neural stem, precursor and progenitor cells. Thus, the negative selection agent may select against differentiated cells with a non-DA phenotype, such as non-DA neurons. The negative selection agent may reduce or prevent proliferation of and/or kill cells other than the desired cell type(s). The negative selection agent may be a selective neurotoxin that reduces the population of neurons other than DA neurons. For example, the negative selection agent may be 5-7-dihydroxytryptamine (to reduce serotoninergic neurons). The negative selection agent may be an antibody or antibody fragment specific for a non-DA neuron, wherein the antibody or antibody fragment (e.g. scFv or Fab) is coupled to saponin or to a toxin. For instance the antibody may be specific for GABA transporter (to reduce GABAergic neurons).

In methods of the invention, the neural stem, progenitor or precursor cell or other stem or neural cell may be grown in the presence of an antioxidant (e.g. ascorbic acid), low oxygen tension and/or a hypoxia-induced factor (e.g. HIF or erythropoetin).

The present invention further provides in various aspects and embodiments the use of a substance the provides inhibition of GSK-3β, e.g. a GSK-3β inhibitor, in therapeutic methods comprising administering the substance or, if the substance is peptidyl in nature (a peptide, polypeptide or protein), encoding nucleic acid to an individual to induce, promote or enhance dopaminergic neuron development in the brain by acting on either endogenous or on exogenously supplied stem, progenitor or precursor cells, or neuronal cells, and/or to inhibit or prevent loss or promote the survival or phenotypic differentiation or maturation, or neuritogenesis or synaptogenesis, or functional output, of dopaminergic neurons, e.g. in treatment of an individual with a Parkinsonian syndrome or Parkinson's disease. A substance may be administered in any suitable composition, e.g. comprising a pharmaceutically acceptable excipient or carrier, and may be used in the manufacture of a medicament for treatment of a neurodenerative disorder, Parkinsonian syndrome or Parkinson's disease. A substance or encoding nucleic acid may be administered to or targeted to the central nervous system and/or brain.

The present invention extends in various aspects not only to a neuron produced in accordance with any one of the methods disclosed herein, but also a pharmaceutical composition, medicament, drug or other composition comprising such a neuron, stem, progenitor or precursor cell and/or substance that provides regulation of inhibition of GSK-3β, e.g. GSK-3β inhibitor, use of such a neuron, stem, progenitor or precursor cell or neuronal cell and/or substance or composition in a method of medical treatment, a method comprising administration of such a neuron, stem, progenitor, precursor or neuronal cell and/or substance or composition to a patient, e.g. for treatment (which may include preventative treatment) of Parkinson's disease or other (e.g. neurodegenerative) diseases, use of such a neuron or cell and/or substance in the manufacture of a composition for administration, e.g. for treatment of Parkinson's disease or other (e.g. neurodegenerative diseases), and a method of making a pharmaceutical composition comprising admixing such a neuron or cell and/or substance with a pharmaceutically acceptable excipient, vehicle or carrier, and optionally one or more other ingredients, e.g. a neuroprotective molecule, a neuroregenerative molecule, a retinoid, growth factor, astrocyte/glial cell, anti-apoptotic factor, or factor that regulates gene expression in stem, progenitor or precursor cells or neuronal cells or in the host brain. Such optional ingredients may render the neuron independent of its environment, i.e. such that its survival is not dependent on the presence of one or more factors or conditions in its environment. By way of example, the method of making a pharmaceutical composition may include admixing the neuron with one or more factors found in the developing ventral mesencephalon. The neuron may be admixed with GDNF and/or neurturin (NTN). The method of making a pharmaceutical composition may include expressing BcLXL in the neuron with one or more factors found in the developing ventral mesencephalon.

The present invention provides a composition containing a neuron, stem, progenitor or precursor cell or neuronal cell produced in accordance with the invention and/or substance that acts in GSK-3β inhibition, e.g. GSK-3β inhibitor, and one or more additional components. Pharmaceutical compositions according to the present invention, and for use in accordance with the present invention, may comprise, in addition to the neuron or cell, a pharmaceutically acceptable excipient, carrier, buffer, preservative, stabiliser, anti-oxidant or other material well known to those skilled in the art. Such materials should be non-toxic and should not interfere with the activity of the neuron. The precise nature of the carrier or other material will depend on the route of administration. The composition may include one or more of a neuroprotective molecule, a neuroregenerative molecule, a retinoid, growth factor, astrocyte/glial cell, or factor that regulates gene expression in stem, neural stem, precursor or progenitor cells or neuronal cells. Such substances may render the neuron independent of its environment as discussed above.

Liquid pharmaceutical compositions generally include a liquid carrier such as water, petroleum, animal or vegetable oils, mineral oil or synthetic oil. Physiological saline solution, tissue or cell culture media, extracellular matrix protein, heparan or chondroitin sulfate, dextrose or other saccharide solution or glycols such as ethylene glycol, propylene glycol or polyethylene glycol may be included.

The composition may be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition may be prepared using artificial cerebrospinal fluid.

The present invention extends to the use of a neuron produced in accordance with the invention and/or GSK-3β inhibiting substance, e.g. GSK-3β inhibitor, in a method of medical treatment, particularly the treatment of a medical condition associated with degeneration, damage to, the loss of, or a disorder in neuronal cells. Moreover, the invention may provide the use of a neuron of a specific phenotype and/or substance that regulates or provides GSK-3β inhibition, e.g. GSK-3β inhibitor, in the treatment of a condition, disease or disorder, which is associated with generation, damage to, or the loss of neurons of that phenotype. More particularly, the invention provides the use of a dopaminergic neuron and/or GSK-3β inhibitory substance in the treatment of human Parkinson's disease. While the invention particularly relates to materials and methods for treatment of neurodegenerative diseases (e.g. Parkinson's disease), it is not limited thereto. By way of example, the invention extends to the treatment of degeneration in or damage to the spinal cord and/or cerebral cortex, or other regions of the nervous system containing Nurr1+ cells.

In methods of treatment in which the administered cell is a stem, progenitor or precursor that is capable of giving rise to two or more distinct neuronal phenotypes, the neuron, cell and/or Wnt ligand or composition may be introduced into a region containing astrocytes/glial cells which direct the differentiation of the cell to a desired specific neuronal fate. The cell and/or Wnt ligand or composition may for example be injected into the ventral mesencephalon where it may interact with Type 1 astrocytes/glial cells and be induced to adopt a dopaminergic phenotype. Alternatively or in addition, an implanted composition may contain a neuron or cell in combination with one or more factors which direct its development toward a specific neuronal fate as discussed above, e.g. with a Type 1 astrocyte/glial cell.

Cells may be implanted into a patient by any technique known in the art (e.g. Lindvall, O., (1998) Mov. Disord. 13, Suppl. 1:83-7; Freed, C. R., et al., (1997) Cell Transplant, 6, 201-202; Kordower, et al., (1995) New England Journal of Medicine, 332, 1118-1124; Freed, C. R., (1992) New England Journal of Medicine, 327, 1549-1555). See also Freed et al. J. Neurol. 2003 October; 250 Suppl 3:III44-6; Dunnett et al. (2001) Nat Rev Neurosci. 2001, 2(5):365-9; Lindvall and Hagell (2000) Prog Brain Res. 2000, 127:299-320; Mendez et al. J. Neurosurg. 2000 May; 92(5):863-9; Hagell et al. Mov Disord. 2000 March; 15(2):224-9; Hauser et al. Arch Neurol. 1999 February; 56(2):179-87; Lindvall Mov Disord. 1998; 13 Suppl 1:83-7. Review.; Olanow et al. Trends Neurosci. 1996 March; 19(3):102-9. Review; Remy et al. Ann Neurol. 1995 October; 38(4):580-8; Kordower et al. N Engl J. Med. 1995 Apr. 27; 332(17):1118-24; Lindvall et al. Ann Neurol. 1994 February; 35(2):172-80; Hoffer et al. Exp Neurol. 1992 December; 118(3):243-52; Lindvall et al. Arch Neurol. 1989 June; 46(6):615-31.

Administration of a composition in accordance with the present invention is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

The methods provided herein may be carried out using primary cells in vivo or in vitro or cell lines as a source material. The advantage of cells expanded in vitro is that there is virtually no limitation on the number of neurons which may be produced.

In order to ameliorate possible disadvantages associated with immunological rejection of transplanted cells, stem or progenitor or precursor cells may be isolated from a patient and induced to the desired phenotype. Cells may then be transplanted to the patient. Advantageously, isolated stem or progenitor or precursor cells may be used to establish cell lines so that large numbers of immunocompatible neuronal cells may be produced. A further option is to establish a bank of cells covering a range of immunological compatibilities from which an appropriate choice can be made for an individual patient. Stem, neural stem, precursor or progenitor cells or neuronal cells derived from one individual may be altered to ameliorate rejection when they or their progeny are introduced into a second individual. By way of example, one or more MHC alleles in a donor cell may be replaced with those of a recipient, e.g. by homologous recombination. Immunosupresive treatment may be administrated to the patients.

If cells derived from a cell line carrying an immortalizing oncogene are used for implantation into a patient, the oncogene may be removed using the CRE-loxP system prior to implantation of the cells into a patient (Westerman, K. A. et al Proc. Natl. Acad. Sci. USA 93, 8971 (1996)). An immortalizing oncogene which is inactive at the body temperature of the patient may be used.

Stem, neural stem, precursor, progenitor or neural cells which may be used in the present invention include embryonic stem cells, fetal stem cells, adult bone marrow or hemotopoyetic stem cells, mesangioblasts, and human cell lines such as H6 (Flax et al. Nature Biotech 16 (1998)). Further examples are listed in: Gage et al. Ann. Rev. Neurosci. 18, 159-192 (1995) and Gottlieb 2002).

While the present discussion has been made with reference to neural stem cells or neural progenitor or precursor cells, the methods provided herein may be applied to the induction of neuronal fates in other stem, progenitor or precursor cells. Examples of such cells include stem cells associated with non-neural systems. The methods may be applied to stromal or hematopoietic stem cells and/or proliferative cells from the epidermis. Hematopoietic cells may be collected from blood or bone marrow biopsy. Stromal cells may be collected from bone marrow-biopsy. Epithelial cells may be collected by skin biopsy or by scraping e.g. the oral mucosa. Since a neuronal phenotype is not a physiological in vivo fate of these stem, progenitor or precursor cells, the inductive process may be referred to as trans-differentiation, or de-differentiation and neural re-differentiation. A method of inducing such cells to a neuronal fate may include the use of antisense regulators to genes associated with non-neuronal phenotypes, i.e. to suppress and/or reverse the differentiation of these cells toward non-neuronal fates.

The methods of the present invention may be applied to stem cells not committed to a neural fate. They may be applied to stem cells which are capable of giving rise to two or more daughter stem cells associated with different developmental systems. Examples of these stem cells are embryonic stem cells, hematopoietic stem cells, bone marrow stromal stem cells, proliferative cells from the epidermis, and neural stem cells.

As discussed above, the present disclosure demonstrates that dopaminergic neurons can be generated from stem or progenitor or precursor cells by a process requiring expression of Nurr1 above basal levels in combination with GSK-3β inhibition and/or one or more factors derived from ventral mesencephalic type 1 astrocytes or glial cells.

In various further aspects the present invention is concerned with provision of assays and methods of screening for a factor or factors which enhance induction of a dopaminergic fate in a neural stem or progenitor or precursor cell or enhance dopaminergic induction or differentiation in a neuronal cell expressing Nurr1 above basal levels by acting as a means of regulating inhibition of GSK-3β, e.g. as a GSK-3β inhibitor or means to inhibit GSK-3β indirectly, e.g. by causing overexpression or stabilisation or inhibition of degradation of β-catenin or inhibition of β-catenin phosphorylation, and with a factor or factors identified thereby.

The invention provides a method of screening for a factor or factors able to inhibit or otherwise regulate inhibition of GSK-3β, either directly or indirectly, and able, either alone or in combination, to enhance, increase or potentiate induction of a dopaminergic fate in a stem, neural stem or progenitor or precursor cell or neuronal cell expressing Nurr1 above basal levels. A further aspect of the present invention provides the use of a stem, neural stem or progenitor or precursor cell or neuronal cell expressing Nurr1 above basal levels inhibitor in screening or searching for and/or obtaining/identifying a means of inhibiting or regulating inhibition of GSK-3β which enhances induction of a dopaminergic fate in such a stem or progenitor or precursor cell or neuronal cell.

A test substance may be tested initially for ability to regulate inhibition of GSK-3β, e.g. to inhibit GSK-3β, whether a direct GSK-3β inhibitor or able to cause overexpression or stabilisation or inhibition of degradation of β-catenin or inhibition of β-catenin phosphorylation. Once identified has having the ability, the substance may be tested for ability to enhance, increase or potentiate induction of a dopaminergic fate in a stem, neural stem or progenitor or precursor cell or neuronal cell expressing Nurr1 above basal levels, as disclosed herein.

As part of a screening method, a test substance may be tested initially for binding or interaction with GSK-3β or substrates, including β-catenin, glycogen synthase, tau, elf2B, CREB or c-jun.

Binding or interaction may be determined by any number of techniques known in the art, qualitative or quantitative. Interaction between a test substance and a stem or progenitor or neuronal cell may be studied by labeling either one with a detectable label and bringing it into contact with the other which may have been immobilised on a solid support, e.g. by using an antibody bound to a solid support, or via other technologies which are known per se including the Biacore system.

A screening method may include culturing a stem, neural stem or progenitor or precursor cell or neuronal cell in the presence of a test substance or test substances and analyzing the cell for differentiation to a dopaminergic phenotype, e.g. by detecting a marker of the dopaminergic phenotype as discussed herein. Tyrosine hydroxylase (TH) is one marker of the dopaminergic phenotype. Other dopaminergic markers include the expression of dopamine transporter (DAT), dopamine receptors, amino acid decarboxylase (AADC), the nuclear receptor Nurr1, the homeoboxes Ptx3 and Lmxlb. Neuronal markers such as neurofilament or beta-tubulin-III should also be expressed by the cells in addition to one of the previous markers. Other methods to detect dopaminergic neurons include detection of dopamine release, and reporter systems for instance TH promoter coupled to luciferase of GFP, etc.

It should be noted that these markers and methods for detection of dopaminergic neurons are of use in determination of successful induction of dopaminergic fate in any of the aspects and embodiments of the present invention.

Any of the substances screened in accordance with by the present invention may be a natural or synthetic chemical compound.

Phosphorylation by GSK-3β as a measure of activity may be determined using any of variety of techniques available in the art.

In a further aspect of the invention there is provided an assay method which includes:

(a) bringing into contact a substrate for GSK-3β including a phosphorylation site, e.g. Ser652 and Ser 648 in Glycogen synthase; Ser37 and Thr41 in beta-catenin; Ser535 in elF2B; Thr129 in CREB, or Thr239 in c-JUN and a test substance in the presence of GSK-3β under conditions in which GSK-3β normally phosphorylates said substrate; and

(b) determining phosphorylation of said substrate. Such a method allows for a substance that acts in GSK-3β inhibition to be identified.

Phosphorylation may be determined for example by immobilising a substrate, e.g. on a bead or plate, and detecting phosphorylation using an antibody or other binding molecule which binds the relevant site of phosphorylation with a different affinity when the site is phosphorylated from when the site is not phosphorylated. Such antibodies may be obtained by means of any standard technique, e.g. using a phosphorylated peptide. Binding of a binding molecule which discriminates between the phosphorylated and non-phosphorylated form of the substrate may be assessed using any technique available to those skilled in the art, which may involve determination of the presence of a suitable label, such as fluorescence. Phosphorylation may be determined by immobilisation of the substrate e.g. on a bead or plate impregnated with scintillant, such as in a standard scintillation proximetry assay, with phosphorylation being determined via measurement of the incorporation of radioactive phosphate. Phosphate incorporation into a substrate may be determined by precipitation with acid, such as trichloroacetic acid, and collection of the precipitate on a suitable material such as nitrocellulose filter paper, followed by measurement of incorporation of radiolabeled phosphate. SDS-PAGE separation of substrate may be employed followed by detection of radiolabel.

Other approaches for screening for GSK-3βinhibition include consideration of any one or more of the following:

(i) stabilization or nuclear translocation of wild type β-catenin, or β-catenin-tagged with a label or reporter, e.g. a fluorescent label or enzyme;

(ii) analysing for the regulation of the expression of known β-catenin target genes, some of which contain consensus binding sites for TCF/LEF family members in their regulatory regions. These include but are not limited to: c-myc, Cyclin D, Tcf-1, PPARdelta, c-jun, fra-1, uPAR, matrix metalloproteinase MMP-7, Axin-2, Nr-CAM, ITF-2, Gastrin, CD44, EphB/ephrin-B, BMP4, claudin-1, Survivin, VEGF, FGF18, FoxN1, matrix metalloproteinase-26, Frizzled 7, Follistatin, Id2, siamois, twin, Xnr3, fibronectin, myogenic bHLH, engrailed-2, connexin43, connexin 30, retinoic acid receptor gamma, dharma/bozozok, Cdx4, nacre, Stra6, Wrch-1, TNF family 41BB ligand, ephrinB1, Stra6, autotaxin and ISLR, Twist, Stromelysin, Brachyury, Proglucagon, Osteocalcin, cyclooxygenase-2, Irx3, Six3, neurogenin 1, WISP-1, WISP-2, IGF-II, Proliferin-2, Proliferin-3, Emp, IGF-I, VEGF-C, MDR1, COX-2, IL-6,Periostin, Cdxl, betaTrCP, sFRP-2, Pitx2, Eda (TNF-related), E-cadherin, Keratin, movol, mBTEB2, FGF4, ret, Ubx, wingless, Dpp, Engrailed-1, Dfrizzled2, shavenbaby, stripe, Nemo, (see for example the Stanford website describing targets in pathways, findable using any web browser and at http://www.stanford.edu/˜rnusse/pathways/targets.html updated in June 2004). The regulation of these genes may be monitored by a wide variety of techniques, including measuring the levels of β-catenin protein with biochemical and/or immunological methods or measuring Nurr1 mRNA levels by hybridization and/or PCR or measuring the level of Nurr1 gene expression using a recombinant promoter/reporter gene expression system. This latter approach would involve expressing within the cells used for drug screening a DNA construct comprising such cis regulatory elements as are needed to regulate the endogenous expression of Nurr1 operably linked to a reporter gene(s).

(iii) regulation of a construct comprising a β-catenin response element or linked to a reporter gene or reporter genes. This allows monitoring of the specific transcription-promoting activity of β-catenin instead of or in conjunction with measuring the expression levels. This allows for more rapid and high-throughput methods for determining whether particular treatments causes GSK-3β inhibition or affects the acquision of a dopaminergic phenotype. Reporter constructs that may be used may include a bioluminescent protein, or enzymatic activity, luciferase chloramphenicol acetyltransferase, beta-galactosidase, beta-lactamase or other antibiotic resistance gene, a neurotrophin or cytokine, and so on.

(iv) Gene discovery. β-catenin activity likely upregulates more than just the TH gene. β-catenin activation probably leads to changes in the expression level of several downstream genes involved in specifying the dopaminergic phenotype. While many such genes may well have previously been identified or characterized, some may be as yet unidentified. Moreover, even if identified, their function in specifying a dopaminergic phenotype may not have been previously recognized. These genes may represent unrecognized targets for therapies to compensate for the loss of dopaminergic neurons. The identification of target genes may be examined by: overexpressing β-catenin constitutively or under an inducible promoter or inducing the expression of endogenous or artificial β-catenin gene in cells (stem, progenitor, precursor, nurr1 expressing or neural cell etc). Analysis can be done by assessing in stimulated and unstimulated cells: gene expression, post-translational modifications of known proteins, ion channel function or intracellular localization of particular molecules. Techniques may include subtractive hybridization, differential display, SAGE, biochemical fractionation, electrophysiology or cytochemical techniques. The techniques that are used in the Wnt field include: duplication of axis in Xenopus, somite myogenesis, kidney epithelial induction, axonal remodelling, Neurotrophin-3 expression, aggregation of cardiac myocytes, cell transformation assays, and expansion hematopoietic stem cells (for references see the Stanford website describing assays, findable using any web browser, http://www.stanford.edu/˜rnusse/assays/assays.html updated November 2000).

A screening method may employ any known method for analyzing a phenotypic difference between cells and may be at the DNA, mRNA, cDNA or polypeptide level. Differential screening and gene screening are two such techniques. A substance identified by any of the methods of screening described herein may be used as a test substance in any of the other screening methods described herein.

A screening method may employ a nucleic expression array, e.g. a mouse cDNA expression array. In this approach, an array of different nucleic acid molecules is arranged on a filter, quartz or another surface, e.g. by cross-linking the nucleic acid to the filter. A test solution or extract is obtained and the nucleic acid within it is labeled, e.g. by fluorescence. The solution or extract is then applied to the filter or genechip. Hybridisation of the test nucleic acid to nucleic acid on the filter or genechip is determined and compared to the hybridisation achieved with a control solution. A difference between the hybridisation obtained with the test and control samples is indicative of a different nucleic acid content. For further information on nucleic acid arrays, see Clontech website (e.g. www.clontech.com) or Affymetrix website (e.g. www.affymetrix.com), findable using any available web browser.

Screening methods are described here with reference to Nurr1 expressed above basal levels, but the disclosure also extends to all nuclear receptors of the Nurr1 subfamily e.g. Nor-1 and NGFI-B. Thus, Nurr1 is described by way of example and not by way of limitation. In any method of the invention, a nuclear receptor of the Nurr1 subfamily, including Nurr1 or any other receptor e.g. Nor-1 or NGFI-B, may be expressed above basal levels in the cell.

A screening method may include comparing stem or progenitor or precursor or neural cells with stem or progenitor or precursor cells or neural cells which express Nurr1 above basal levels in the presence of a substance acting in inhibition of GSK-3β, e.g. to identify target genes of Nurr1 and/or a factor or factors which enhance the proliferation and/or self-renewal and/or the differentiation and/or survival and/or promote the acquisition or the induction of a dopaminergic fate and/or induce dopaminergic neuron development in stem, neural stem, precursor, progenitor or neural cells and/or enhance dopaminergic induction or differentiation in a neuronal cell expressing Nurr1 above basal levels in the presence of a substance that provides inhibition of GSK-3β. Once the target gene(s) and/or factor(s) have been identified they may be isolated and/or purified and/or cloned and used in further methods.

A screening method may include purifying and/or isolating a substance or substances from a mixture. The method may include determining the ability of one or more fractions of the mixture to interact with a stem cell, neural stem cell or neural progenitor or precursor cell or neural cell expressing Nurr1 above basal levels in the presence of inhibition of GSK-3β, e.g. the ability to bind to and/or promote the proliferation and/or self-renewal and/or enhance induction, acquisition, differentiation or development of a dopaminergic phenotype or fate in such a stem, neural stem, precursor, progenitor or neural cell. The purifying and/or isolating may employ any method known to those skilled in the art.

A screening method may employ an inducible promoter operably linked to nucleic acid encoding a test substance. Such a construct is incorporated into a host cell and one or more properties of that cell under the permissive and non-permissive conditions of the promoter are determined and compared. The property determined may be the ability of the host cell to induce a dopaminergic phenotype in a stem, neural stem, precursor, progenitor or neural cell expressing Nurr1 above basal levels. A difference in that ability of the host cell between the permissive and non-permissive conditions indicates that the test substance may be able, either alone or in combination, to enhance proliferation and/or self-renewal and/or induction of a dopaminergic fate and/or dopaminergic differentiation, survival or development in a stem, neural stem or progenitor or precursor cell or enhance dopaminergic induction or differentiation in a neuronal cell expressing Nurr1 above basal levels either as or in the presence of inhibition of GSK-3β.

The precise format of any of the screening methods of the present invention may be varied by those of skill in the art using routine skill and knowledge.

A factor or factors identified by any one of the methods provided by the invention may be isolated and/or purified and/or further investigated. It may be manufactured.

In various further aspects, the invention further provides a factor identified by any one of the methods disclosed herein, a pharmaceutical composition, medicament, drug or other composition comprising such a factor (which composition may include a stem, neural stem or progenitor or precursor cell or neuron expressing Nurr1 above basal levels), use of such a factor to enhance induction and/or phenotypic differentiation or maturation and/or survival and/or neuritogenesis and/or synaptogenesis and/or functional output of dopaminergic neurons derived from stem, neural stem or progenitor or precursor cells expressing Nurr1 above basal levels, use of such a factor or composition in a method of medical treatment, a method comprising administration of such a factor or composition to a patient, e.g. for treatment (which may include preventative treatment) of a medical condition associated with degeneration, damage to, loss of, or a disorder in or affecting dopaminergic neurons, e.g. for treatment of Parkinson's disease or another neurodegenerative disease, use of such a factor in the manufacture of a composition, medicament or drug for administration, e.g. for treatment of Parkinson's disease or other (e.g. neurodegenerative diseases), and a method of making a pharmaceutical composition comprising admixing such a factor with a pharmaceutically acceptable excipient, vehicle or carrier, and optionally other ingredients.

In a related aspect, the present invention provides a method of screening for a substance which modulates the ability of a substance that acts directly or indirectly in GSK-3β inhibition to induce a dopaminergic fate in stem, neural stem, precursor or progenitor cells or enhance dopaminergic induction or differentiation in a neuronal cell expressing Nurr1 above basal levels.

Thus, the method may screen for a substance which modulates the ability of an inhibitor of GSK-3β to induce proliferation, self renewal, dopaminergic development, differentiation, maturation and/or acquisition of a dopaminergic fate in stem, neural stem, precursor, progenitor or neural cells expressing Nurr1 above basal levels.

Such a method may include one or more of:
(i) providing stem, neural stem, progenitor, precursor or neural cells which express Nurr1 above basal levels in the presence of inhibition of GSK-3β, e.g. GSK-3β inhibitor or agent able to cause overexpression or stabilisation or inhibition of degradation of β-catenin or inhibition of β-catenin phosphorylation, or binding or interacting with GSK-3βor a GSK-3β substrate, phosphorylation of a GSK-3β substrate, nuclear translocation of β-catenin, regulation of the expression of a β-catenin target gene or a β-catenin response element linked to a reporter gene, and one or more test substances;
(ii) analysing the proportion of such cells which adopt a dopaminergic fate or phenotype and/or respond to the inhibitor;
(iii) comparing the proportion of such cells which adopt a dopaminergic fate with the number of such cells which adopt a dopaminergic fate or phenotype and/or respond to the inhibitor in comparable reaction medium and conditions in the absence of the test substance or test substances. A difference in the proportion of dopaminergic neurons between the treated and untreated cells is indicative of a modulating effect of the relevant test substance or test substances.

Such a method of screening may include:
(i) bringing stem, neural stem, precursor or progenitor cells or neuronal cells which express Nurr1 above basal levels into contact with an inhibitor of GSK-3β (whether direct or indirect inhibitor) in the presence of one or more test substances;
(ii) analysing the proportion of stem, neural stem, precursor or progenitor cells or neuronal cells which adopt a dopaminergic fate or phenotype and/or respond to the inhibitor;
(iii) comparing the proportion of stem, neural stem, precursor, progenitor or neural cells which adopt a dopaminergic fate or phenotype and/or respond to the inhibitor with the number of stem, precursor, progenitor or neural cells which adopt a dopaminergic fate or phenotype and/or respond to Wnts in comparable reaction conditions in the absence or presence of the test substance or test substances.

Such screening methods may be carried out on cells in vivo in comparable or identical non-human animals, or in vitro or in culture.

Following identification of a substance which modulates GSK-3β inhibition, the substance may be investigated further. It may be manufactured and/or used in the preparation, i.e. manufacture or formulation, of a composition such as a medicament, pharmaceutical composition or drug. Any of substance tested for its modulating activity may be a natural or synthetic chemical compound.

A modulator that enhances GSK-3β inhibition, e.g. a enhances activity of a direct GSK-3β inhibitor, for example, may be provided with the inhibitor, and the two substances may be used together, e.g. in a pharmaceutical composition and other embodiments of the present invention in its various aspects.

Means to regulate GSK-3β inhibition may be used in accordance with the invention to restore dopaminergic phenotype, as has been disclosed. In further embodiments, regulation of GSK3 inhibition may be accomplished by administering substances that regulate transcriptional activity of genes involved in GSK-3β inhibition therapy, or by administering genes that regulate GSK-3β inhibition via gene therapy. In vitro we have shown that β-catenin administration, as a mean of providing GSK3 inhibition, results in an increase in the generation of dopaminergic neurons from neural cells. Introducing genes that regulate GSK3 inhibition into neural cells in the brain may be used increase the number of dopaminergic cells in the brains of patients. Alternatively, PD patients may be treated with substances capable of regulating GSK3 inhibition. Such substances may be identified via drug discovery assays of the types described above.

The invention also provides for diagnostic applications. Since GSK3 inhibition promotes the acquisition of a dopaminergic phenotype and loss of that phenotype is associated to Parkinson's disease, abnormalities in GSK-30 inhibition, including altered levels and/or function of dishevelled, axin, FRAT/GBP, casein kinase 1, beta-catenin may underlie the etiology of Parkinson's disease. Structural or functional abnormalities in the genes, mRNA or proteins involved in GSK-3β inhibition may result in abnormal levels and/or function of the gene, mRNA or protein, and these defects and/or abnormalities may be manifest prior or after to the clinical onset of PD. Thus screening for such abnormalities at DNA, mRNA or protein level may provide for presymptomatic detection of PD or may provide a confirmation of diagnosis. These techniques may be applied to brain biopsies, cerebrospinal fluid, blood, saliva, urine or other bodily fluid or tissue.

Aspects and embodiments of the present invention will now be illustrated, by way of example, with reference to the accompanying figures. Further aspects and embodiments will be apparent to those skilled in the art. All documents mentioned in this specification are incorporated herein by reference.

### BRIEF DESCRIPTION OF THE FIGURES

FIG. 1—KP and I3M increase the number of TH+ neurons in VM precursor cultures.

FIG. 1A: Dose-response experiments in E14.5 VM precursors indicate that 15 μM KP and 3-5 μM I3M are optimal doses in order to increase TH+ cell numbers.

FIG. 1B: Treatment of SN4741 cells with 15 μM KP and 5 μM I3M for 24 hours decreases GSK-3β protein levels as assessed by immunoblotting. Loading control: beta-actin, Immunostaining showed an increase in the number of TH immunoreactive cells three days after treatment with 15 μM KP and 5 μM I3M. Statistical analysis was performed using one-way ANOVA with Fisher's pos-hoc test.

FIG. 2—Treatment with KP and I3M increases differentiation of VM precursors into both neurons and glia, and particularly into DA neurons. Immunostainings with antibodies against GFAP (A), Tuj1 and TH (B), and MAP2 (C) showed an increase in the number of astrocytes, immature and mature neurons and a marked increase in the proportion of TH+ DA neurons (B and D) three days after treatment with 15 μM KP and 5 μM I3M. Statistical analysis was performed using two-tailed unpaired t-test.

FIG. 3—GSK-3β inhibition does not regulate cell survival or proliferation of VM precursors or MN9D cells but induces their morphological differentiation.

FIG. 3A shows that treatment with 15 μM KP and 5 μM I3M did not change the total number of VM cells after 3 days in culture, as assessed by Hoechst 33258 staining.

FIG. 3B shows that proliferation, as assessed by BrdU incorporation, was not affected by 15 μM KP, but was significantly decreased with 5 μM I3M.

FIG. 3C shows that active Caspase 3 immunostaining was not modified by 15 μM KP in VM precursor cultures, but was reduced by 5 μM I3M.

FIG. 3D shows that treatment of MN9D cells with 15 μM KP for four days did not alter the total number of cells (Hoechst 33258) or their proliferation (BrdU incorporation). Immunostaining with α-active caspase 3 showed a decreased number of positive cells three days after treatment with 5 μM I3M, but not with KP. Treatment with 15 μM KP for four days induced morphological differentiation of MN9D cells. Statistical analysis was performed using two-tailed upaired t-test (A), Kruskal-Wallis test (B), one-way ANOVA with Boneferroni's multiple comparison test (C).

FIG. 4—KP up-regulates c-ret mRNA and increases conversion of Nurr1+ precursors into TH+ neurons.

FIG. 4A shows that real time RT-PCR analysis revealed that c-ret mRNA levels were upregulated upon treatment with 15 μM KP.

FIG. 4B shows that 15 μM KP increased theconversion of VM Nurr1+ precursors into TH+ neurons.

TH/Nurr1 double immunostaining showed an increase in the number of immunoreactive cells three days after treatment with 15 μM KP. Statistical analysis was performed using a two-tailed Wilcoxon's signed rank test (A) and a two-tailed unpaired t-test (B).

FIG. 5—GSK-3β inhibitors stabilize β-catenin and overexpression of β-catenin increases the number of TH+ neurons.

FIG. 5A shows results of treatment of VM precursors with 15 μM KP or 5 μM I3M for 24 hours increased both β-catenin and TH protein levels, as assessed by immunoblotting. Loading control: beta-actin.

FIG. 5B reports on TH-immunostaining showing a two-fold increase in the number of positive cells two days after transfection with β-catenin, compared to transfection with an empty control vector. Statistical analysis was performed using a two-tailed unpaired t-test.

Specifically incorporated by reference herein are the experimental results set out in WO00/66713 demonstrating proliferation and/or self-renewal of dopaminergic precursors and induction of dopaminergic neurons in stem, neural stem, precursor or progenitor cells expressing Nurr1, in the presence of type 1 astrocytes or glial cells, and demonstrating additional results obtained when contacting such cells with additional factors, such as FGFs (e.g. FGF8) or retinoids.

Also specifically incorporated herein by reference are the experimental results set out in WO2004/029229 relating to use of Wnts in dopaminergic neuron differentiation.

### GSK-3β Inhibition Increases the Number of DA Neurons in VM Precursor Cultures.

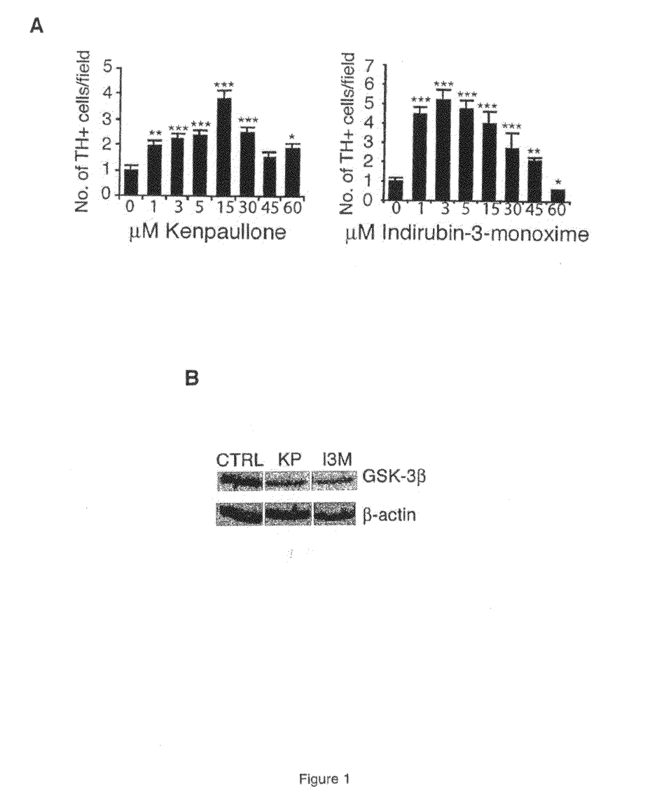

Precursor cells in the rat VM acquire a DA phenotype between embryonic day 11 and 16 (E11-E16) (16). As proliferating precursors in the neuroepithelium begin migrating ventrally, they start expressing Nurr1 (17), thereby withdrawing from the cell cycle and acquiring a DA phenotype by a mechanism involving the cyclin dependent kinase (CDK) inhibitor p57 (18). We have previously shown that Wnts promote DA differentiation in VM precursors (7). In order to investigate whether intracellular canonical Wnt signaling components can modulate DA neuron development, we treated rat VM E14.5 precursor cultures with increasing doses of two ATP competitive inhibitors of GSK-3β, a critical enzyme for β-catenin degradation. Addition of Indirubin-3-monoxime (I3M) (19, 20) or Kenpaullone (KP) (13) to precursor cultures increased the number of TH positive cells in a dose-dependent manner, with maximal effects at 3-5 μM for I3M (five-fold increase) and 15 μM for KP (three-fold increase) (FIGS. 1A and 1C). At these concentrations these compounds differ in that KP is a GSK-3β selective inhibitor while I3M inhibits both GSK-3β and CDKs (20, 13). Interestingly, we also found that treatment of a DA precursor cell line derived from the substantia nigra, SN4741 (21) with KP or I3M led to a downregulation of GSK-3β protein levels (FIG. 1B).

### GSK-3β Inhibition Increases Differentiation of VM Precursors

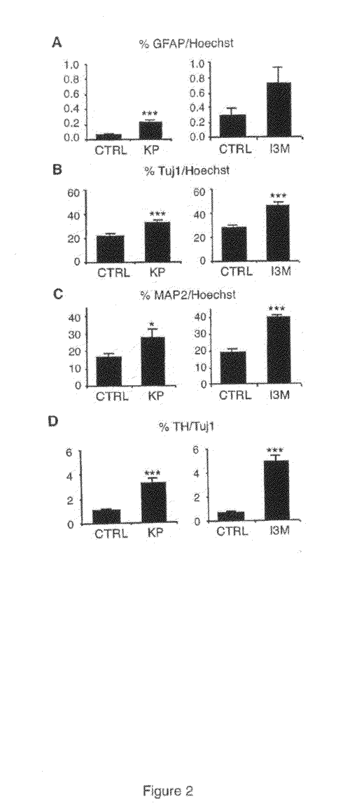

We next asked whether inhibition of GSK-3β affected general neuronal and glial differentiation of VM precursors. We found that both I3M and KP increased the total number of astrocytes (glial fibrillary astrocytic protein (GFAP) positive cells/Hoechst 33258) (FIG. 2A), and immature neurons (beta-tubulin type III (TuJ1) positive cells/Hoechst 33258) (FIG. 2B), as assessed by 6 immunocytochemistry (FIGS. 2A and 2B). Significantly, GSK-3β inhibition also lead to an increased number of mature neurons (microtubule-associated protein 2 (MAP2) positive cells/Hoechst 33258) (FIG. 2C) and to a very pronounced increase in DA neuron differentiation, as shown by a 3 to 5-fold increase in the proportion of TH+/TuJ1+ cells in the VM (FIG. 2D and images in FIG. 2B). Interestingly, we have previously suggested that the differentiation of Nurr1+ stem/precursor cells into DA neurons depends on signals derived from glia (22, 23). Thus, it is possible that the increased differentiation of glial cells could also contribute to the enhanced differentiation of DA neurons.

GSK-3β does not Regulate Cell Survival or Proliferation in VM Precursor Cultures.

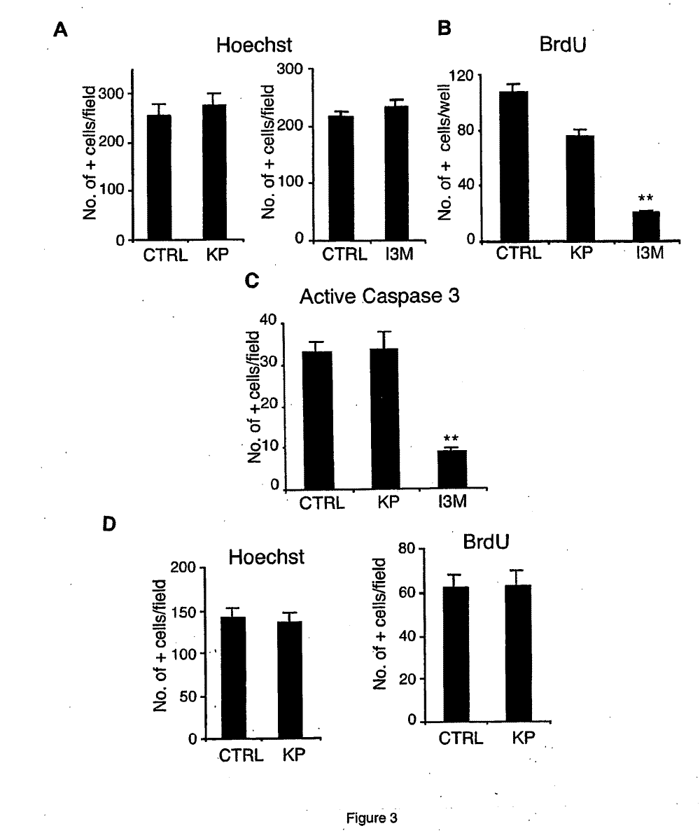

In order to determine whether GSK-3β inhibition was promoting proliferation, we examined the total number of cells in the culture and the number of cells that incorporate BrdU, a marker of proliferation. Treatment with the GSK-3β inhibitors did not change the total number of cells present in the culture after 3 days, as assessed by Hoechst 33258 staining (FIG. 3A). Moreover, when VM precursors were exposed to a 6 h BrdU pulse before fixing, KP did not significantly affect BrdU incorporation (FIG. 3B). However, consistent with previous reports showing that I3M inhibits CDKs (20, 13), we found that I3M treatment reduced the number of BrdU+ cells (FIG. 3B). Thus, our results suggest that the increase in TH+ neurons by GSK-3β inhibition was not achieved through proliferation. We next examined whether GSK-3β inhibitors could increase the number of DA cells through survival. While the GSK-3β/CDK inhibitor, I3M, reduced the number of active Caspase-3 immunoreactive cells, KP, the selective GSK-3βinhibitor, had no effect (FIG. 3C-D). More importantly, we found that about 5 percent of the TH+ cells stained positive for active Caspase 3 but neither of the two GSK-3β inhibitors 7 decreased the number of Caspase+/TH+ cells (data not shown), indicating that GSK-3β inhibition does not lead to increased numbers of TH+ neurons through decreased apoptosis. In order to confirm the effects of GSK-3β inhibition on DA differentiation, we investigated the effects of the selective GSK-3β inhibitor KP on MN9D cells that exhibit features of immature DA neurons (24). As in the VM precursor cells, treatment with KP did not change the total number of cells (assessed by Hoechst 33258) or the level of BrdU incorporation (FIG. 3E). KP treatment did however increase morphological differentiation and neurite extension of MN9D cells (FIG. 3F), similar to the findings in the VM precursor cultures (FIG. 2C). Comparable effects on morphological differentiation of MN9D cells have been reported after Nurr1 overexpression (18), suggesting that these cells and VM precursors share the capacity to differentiate in response to Nurr1 and GSK-3β inhibitors. Combined, our results suggested a function of GSK-3β in precursor cell differentiation and prompted us to examine whether GSK-3β inhibition promotes DA differentiation in VM precursors.

Inhibition of GSK-3β Increases DA Cell Numbers Through Conversion of Nurr1+ Precursors into DA Neurons

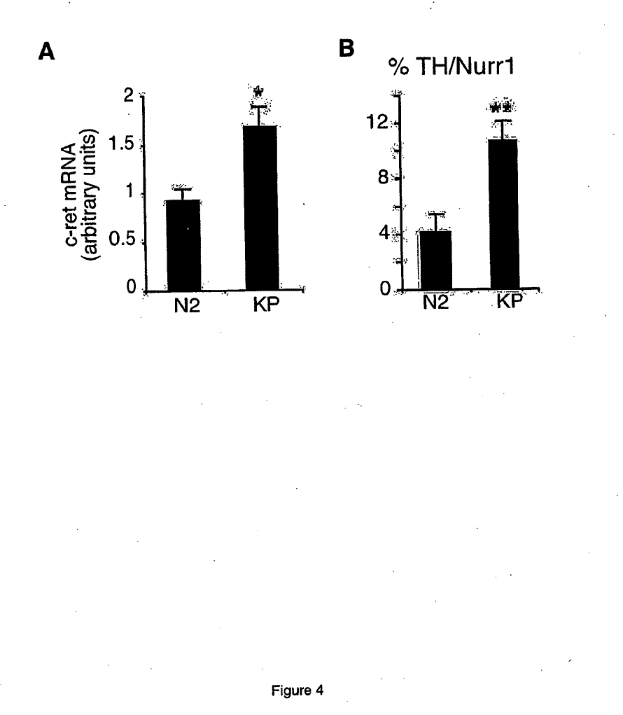

We first asked whether GSK-3β inhibition promoted the expression of genes characteristic of differentiated VM DA neurons. Real-time RT-PCR analysis showed that KP treatment resulted in increased mRNA levels of the protooncogene c-ret (FIG. 4A). C-ret has also been reported to be upregulated upon treatment of VM precursor cultures with Wnt5a (7) or in catecholaminergic PC12 cells treated with Wntl (25). This result suggested that GSK-3β inhibition might work to increase the differentiation of DA precursors. We therefore examined the differentiation of Nurr1+/TH-precursors into Nurr1+/TH+ DA neurons upon treatment with KP and found that this 8 specific GSK-3β inhibitor increased the number of TH/Nurr1 double positive cells 2.5 fold (FIG. 4B-C). Thus, our results indicate that the mechanism by which GSK-3β inhibition leads to increased numbers of TH+ neurons is not by promoting proliferation or survival of VM precursors but by promoting the differentiation of Nurr1+ VM Precursors.

β-Catenin Stabilization Increases the Differentiation of VM Precursors into DA Neurons

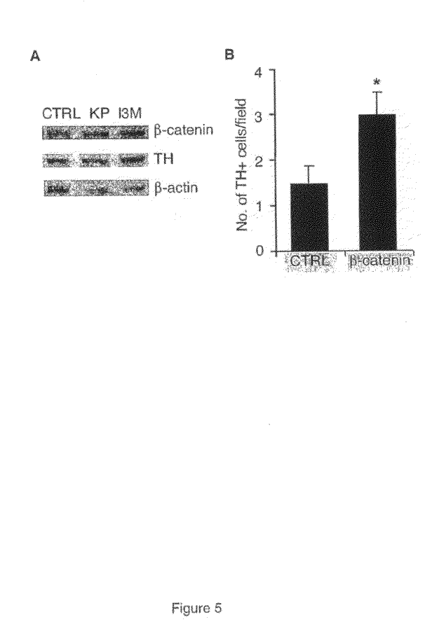

We have previously shown that Wnts promote DA differentiation in VM cultures and that there is Nurr1/β-catenin co-localization and TCF/LEF transcriptional activity in E10.5 VM precursors in vivo (7). However, GSK-3β was recently described to mediate axonal remodeling through microtubule stabilization in a β-catenin independent pathway (26). Thus, in order to further investigate the mechanism of DA differentiation, we analysed the level of β-catenin protein in VM precursor cultures treated with KP or I3M. Immunoblotting showed increased levels of the β-catenin protein, indicating increased stabilization of β-catenin upon treatment with the inhibitors (FIG. 5A). Furthermore, both inhibitors increased the levels of TH protein (FIG. 5A). These results suggested that β-catenin could mediate some of the effects of the GSK-3β inhibitors and Wnts on DA precursors. In order to assess the role of β-catenin stabilization in early DA differentiation, we transfected VM precursors with a β-catenin expressing vector. Overexpression of β-catenin in VM precursor cells led to a two-fold increase in the number of TH positive cells, compared to an empty control vector (FIGS. 5B and C).9

### Methods

### Real-Time PCR and Quantification of Gene Expression

Total RNA was isolated from E14.5 VM precursor cultures (2.5×106 cells in 6 cm2 dishes) treated with the GSK-3β inhibitors for three days in vitro. For a description of reverse transcription reaction, real-time RT-PCR and c-ret primer sequences, see Castelo-Branco et al., 2003 (7). Quantum RNA classical 18S internal standard was purchased from Ambion (Austin, USA) and PCR primers from DNA Technology A/S, Aarhus, Denmark. The following PCR programme was used for SYBR Green detection on the ABI PRISM 5700 Detection System (PE AppliedBiosystems, Foster City, Calif., USA): 94° C. for 2 min, 35-40 cycles of 94° C. for 30 s, 59° C. for 30s, 72° C. for 15s and 80° C. for 5s. Statistical analysis of the results was performed by a 2-tailed Wilcoxon signed rank test. Significance for all tests was assumed at the level of p<0.05 (*p<0.05; **0.01<p<0.001; ***p<0.001).

### Precursor and MN9D Cultures and Treatments

E14.5 VMs obtained from time-mated Sprague-Dawley rats (ethical approval for animal experimentation was granted by Stockholms Norra Djurförsöks Etiska Nämnd) were dissected, mechanically dissociated, and plated at a final density of 1×105 cells per cm2 on poly-D-lysine coated plates (Falcon) in serum-free N2 medium, consisting of a 1:1 mixture of F12 and MEM (Invitrogen) with 5 μg/ml insulin, 100 μg/ml transferrin, 100 μM putrescine, 20 nM progesterone, 30 nM selenium, 6 mg/ml glucose and 1 mg/ml BSA (all purchased from Sigma). Cultures were kept three days in vitro, in a 37° C. incubator, at 5% CO2, before fixation. MN9D and SN4741 cells were grown as described in Choi et al., 1991 and Son et al., 1999. MN9D cultures were fixed after four days in vitro. I3M and KP (Alexis Biochemicals, Göteborg, Sweden) were diluted in DMSO and added to the cultures at the time of plating. DMSO alone did not increase the number of TH+12 neurons and did not affect morphological differentiation of MN9D (data not shown). 10 μM BrdU was added to the culture media 6 hours prior to fixation.

### Constructs and Transfections

Human β-catenin cDNA (a gift from Dr. Steven Byers) was subcloned into the pCAIP2 vector containing the chicken beta-actin promoter under the CMV enhancer. An empty pCAIP2 vector was used as control. Primary cells were transfected using Lipofectamine 2000 (Invitrogen) according to manufacturer's recommendations and assessed by immunocytochemistry after two days in vitro.

### Immunocytochemistry

Cells were fixed in ice-cold 4% paraformaldehyde for 20 minutes and washed in PBS. For a detailed protocol of immunocytochemistry, see Castelo-Branco et al., 2003 (7). The following primary and secondary antibodies were used: mouse α-TH (1:1000 dilution—Immunostar), rabbit α-TH (1:250-Pelfreeze), mouse α-β-III-tubulin (1:1000-Promega), rabbit α.Nurr1 (1:1000-Santa Cruz), mouse α-MAP2 (1:200—Sigma), rabbit A-GFAP (1:400-Dako), mouse α.active-caspase III (1:100-Cell Signaling), mouse α-BrdU (1:100-Dako) and rat α.BrdU (1:100-Abcam), biotinylated 1:500, Cyanine-2 or rhodamine-coupled horse-α-mouse IgG 1:200 or goat α-rabbit IgG 1:200, from Vector and Jackson Laboratories). At the end of the staining procedure, the cultures were incubated with Hoechst 33258 reagent for 10 minutes. BrdU immunocytochemistry included an incubation for 30 minutes with 2NHCL. Images were acquired from stained cells in PBS at room temperature with a Zeiss Axioplan 100M microscope (LD Achrostigmat 20×, 0.3 PH1. 0-2 and LD Achroplan 40×, 0.60 Korr PH2. 0-2) and collected with a Hamamatsu camera C4742-95 (with QED imaging software).

### Immunoblotting

Cell were lysed for 15 min in a modified RIPA buffer containing 20 mM Tris (pH 7.5), 140 mM NaCl, 10% glycerol, 1% IGEPAL, 1 mM β-glycerol-phosphate, 1 mM Na3VO4 and complete protease inhibitors (Roche). Plates were scraped and the cell lysate was centrifuged in a microfuge for 5 min. The supernatant was transferred to a clean tube for protein measurement using the BCA kit (PIERCE) and stored in Laemmli buffer. Equal amounts of protein were analysed by polyacrylamide gel electrophoresis (10% polyacrylamide). The proteins were transferred onto a polyvinylidene difluoride membrane. After blocking in PBS. with 0.1% Tween and 3% BSA, the membrane was incubated with the following primary antibodies overnight at 4° C.: (mouse α-β-catenin 1:500, BD; rabbit α-GSK-3β, 1:1000, Cell Signaling; rabbit α-TH 1:500, PelFreeze and mouse α-β-actin 1:500, Abcam). After washing, the membrane was incubated with an alkaline phosphatase conjugated secondary α-mouse antibody (1:10 000, Pharmacia Amersham) for 1 hr at room temperature and subsequently developed according to manufacturer's instructions for enhanced chemiluminescence.

### Statistical Analysis

Quantitative immunocytochemical data represent the means±standard errors of counts from ten non-overlapping fields, in three wells per condition from three to five separate experiments. For the RT-PCR experiments, five separate experiments were analysed and for the GSK-3β inhibitors dose response, two sets of experiments were analysed. Statistical analysis was performed in Prism 4 (GraphPad software, San Diego, USA) as described in the figure legends, with 14 significance for all tests assumed at the level of p<0.05 (*p<0.05; **0.01<p<0.001; ***p<0.001). Statistical tests were chosen according to the distribution of the sample population.

### REFERENCES

Airaksinen M S, Saarma M. (2002) Nat Rev Neurosci. 3, 383-394
Akerud et al. J. Neurosci. 21, 8108-18 (2001).
Arenas 2002. Brain Res Bull.; 57 (6):795-808. (ref 28)
Baek S H et al., (2003) Proc Natl Acad Sci USA. 100, 3245-3250
Bain et al. 2003. Biochem J.; 371 (Pt 1): 199-204. (ref 13)
Bain et al., 1995, Dev. Biol., 168, 342-357
Baker et al. Genes Dev 1999 Dec. 1; 13(23):3149-59
Bamji et al. 2003. Neuron; 40 (4):719-731 (ref 11)
Barberi et al. 2003 Nat. Biotechnol. 21 (10):1200-1207 (ref 33)
Bennett et al. 2002. J Biol. Chem.; 277 (34):30998-31004 (ref 14)
Bjorklund A, Lindvall O. Nat. Neurosci. 2000 June; 3(6):537-44.
Bjorklund et al., 2002, Proc Natl Acad Sci USA. February 19, 99(4): 2344-9
Bradley R S, Brown A M. EMBO J. 9(5):1569-75 (1990).
Bradley R S, Brown A M. Mol Cell Biol 15(8):4616-22 (1995).
Brustle et al., 1999, Science 285, 754-756.
Capela and Temple, 2002, Neuron 35, 865-875.
Castelo-Branco et al. 2003. Proc Natl Acad Sci U S A. 100 (22): 12747-12752. (ref 7)
Castillo (1997) Genomics, 41, 250-257
Castillo et al. Mol Cell Neurosci 1998 May; 11 (1-2):36-46
Castro et al. 2001. J Biol Chem.; 276 (46):43277-43284 (ref 18)
Chambers et al., 2003, Cell May 30; 113(5):643-55. Chem et al. 2002. Science; 297 (5580):365-369. (ref 34)
Choi et al. 1991 Brain Res.; 552 (1):67-76. (ref 24)
Ciani et al. 2004. J Cell Biol.; 164 (2):243-253 (ref 26)
Cohen et al. 2001. Nat Rev Mol Cell Biol.; 2 (10):769-776. (ref 12)
Conacci-Sorrell et al. (2002) Genes Dev. 16, 2058-2072.
Danielian et al. 1996. Nature; 383 (6598):332-334. (ref 3)
Dann C E, et al., (2001) Nature 412, 86-90.
Deacon et al., 1998, Exp. Neurol., 149, 28-41.
Dorsky et al. 1998. Nature; 396 (6709): 370-373. (ref 1)
Dunnett et al. 2001. Nat Rev Neurosci.; 2 (5):365-369. (ref 27)
Foster et al. Int J Dev Neurosci 1988; 6(4):367-86
Garcia-Castro et al. Science. 2002 Aug. 2; 297(5582):848-51
Gossen, et al., (1995) Science, 268, 1766-1769
Gottlieb D I. Annu Rev Neurosci. 2002; 25:381-407.
Granholm A C, Reyland M, Albeck D, Sanders L, Gerhardt G,
Hoernig G, Shen L, Westphal H, Hoffer B. (2000) J Neurosci. 20, 3182-3190.
Hall A C, Lucas F R, Salinas P C. (2000) Cell 100, 525-535.
Hall et al. 2003. Glia.; 43 (1):47-51. (ref 23)
Hartmann C, Tabin C J. Cell 2001 Feb. 9; 104(3):341-51
Hoessel et al. 1999. Nat Cell Biol.; 1 (1): 60-67. (ref 19)
Hornstein et al. 2004. J Neurosci Res.; 76 (2):169-173. (ref 30)
Houart et al. Neuron. 2002 Jul. 18; 35(2):255.
Hsieh et al. Proc Natl Acad Sci U S A. 96(7):3546-51 (1999).
Huelsken et al. 2001. Cell.; 105 (4): 533-545 (ref 38)
Huelsken et al. 2002. J Cell Sci.; 115 (Pt 21):3977-3978. (ref 9)
Kiecker et al. Development 2001 November; 128(21):4189-201
Kim et al. 2002. Nature. 418 (6893):50-56 (ref 32)
Kioussi C., et al. (2002) Cell 111, 673-685, 38
Kispert et al. Development 1998 November; 125(21):4225-34
Krylova O, et al., (2002) Neuron 35, 1043-1056.
Law, et al., (1992) Mol Endocrinol 6, 2129-2135
Leclerc et al. 2001. J Biol Chem; 276 (1): 251-260. (ref 20)
Lee et al. 2000. Nat Biotechnol.; 18 (6):675-679. (ref 31)
Lee et al. 2004. Science. 303 (5660):1020-1023 (ref 37)
Lindsay 2002; Cur. Op. Pharmacol., 2: 587-594.
Martinez Arias et al., 1999, Curr. Op. Genetics and dev. 9, 447-454.
Marvin et al. Genes Dev 2001 Feb. 1; 15(3):316-27.
Mata de Urquiza et al., Science 2000 Dec. 15; 290: 2140-2144.
McMahon et al. 1990. Cell; 62 (6):1073-1085. (ref 4)
Megason et al. 2002. Development; 129 (9): 2087-2098. (ref 2)
Merrill et al. 2001. Genes Dev.; 15 (13): 1688-1705. (ref 39)
Muroyama et al. Genes Dev. 16(5):548-53 (2002).
Nordstrom et al. Nat Neurosci 2002 June; 5(6):525-32
Nunes I., et al, Proc Natl Acad Sci U S A. 100, 4245-4250 (2003)
Ohmachi et al., 2000; Biochem. Biophys. Res. Comm. 277, 355-360.
Oishi I, et al., 2003, Genes Cells. 2003 July; 8(7):645-54.
Okabe et al., 1996, Mech. Dev., 59, 89-102.
Pandur et al. Nature. 2002 Aug. 8; 418(6898):636-41.
Paratcha G., Ledda F., Ibanez C.F. (2003) Cell 113, in press.
Parr et al. Development 119(1):247-61 (1993).
Patapoutian A, Reichardt L F. (2000) Curr Opin Neurobiol. 10, 392-399.
PCT/IB03/04598
Perrone-Capano et al. 2000. Neuroscience and Biobehavioral Reviews, 24, 119-124 (ref 16)
Pinson et al. 2000. Nature; 407 (6803):535-538. (ref 6)
Price J. & Williams B P. Current Opinion in Neurobiology 11, 564-567 (2001).
Reubinoff et al., 2000, Nat. Biotech., 18, 399-404.
Reubinoff et al., 2001, Nat Biotech., 19, 1134-1140.
Reya T, et al., (2003) Nature 423, 409-414.
Rhinn M, Brand M. Curr Opin Neurobiol 2001 February; 11(1):34-42
Ross et al. Science 2000 Aug. 11; 289(5481):950-3
Rossi F. & Cattaneo E. Nat Rev Neurosci. 2002 May; 3(5):401-9.
Saneyoshi et al. Nature 417(6886):295-9 (2002).
Sato et al. 2004. Nat Med; 10 (1): 55-63. (ref 15)
Saucedo-Cardenas et al. Proc Natl Acad Sci U S A 1998 Mar. 31; 95(7):4013-8
Schaar et al. 1993 Exp Neurol.; 124 (2):368-371. (ref 36)
Schneider et al. Genes Dev 2001 February 1; 15(3):304-15
Schuldiner et al., 2000, PNAS. U S A 97, 11307-11312.
Schuldiner et al., 2001; Brain Res. 913, 201-5.
Shimizu et al. Cell Growth Differ 1997 December; 8(12):1349-58
Shtutman et al. Proc Natl Acad Sci U S A 96(10):5522-7 (1999).
Smidt et al. Proc Natl Acad Sci U S A. 94(24):13305-10 (1997).
Smidt et al., Nat Neurosci. 3, 337-341 (2000).
Son et al. 1999. J Neurosci; 19 (1): 10-20. (ref 21)
Song et al. Nature 2002 May 2; 417(6884):39-44
Storch et al. 2001. Exp Neurol.; 170 (2):317-325. (ref 20)
Taipale J, Beachy P A. Nature. 2001 May 17; 411(6835):349-54.
Tetsu et al. Nature 398(6726):422-6 (1999).
Thomas et al. 1990. Nature; 346 (6287):847-850. (ref 5)
Tropepe.et al., 2001, Neuron 30, 65-78.
Trupp M et al., (1996). Nature 381, 785-789
Tumbar et al. 2004. Science; 303 (5656):359-363 (ref 40)
Tzahor E, Lassar A B. Genes Dev 2001 Feb. 1; 15(3):255-60
Van Den Munckhof P, et al. 2003 Development 130, 2535-2542
Wagner et al. 1999 Nat. Biotechnol.; 17 (7):653-659. (ref 22)
Wallen et a1.1999. Exp Cell Res; 253 (2): 737-746. (ref 17)
Willert et al. 2003, Nature; 423 (6938):448-452 (ref 8)
Wilson et al. Nature 2001 May 17; 411(6835):325-30
Wurst W, Bally-Cuif L. Nat Rev Neurosci 2001 February; 2(2):99-108
Xing, et al., (1997) Mol Brain Res 47, 251-261
Ye et al. Cell 93(5):755-66 (1998).
Yoshikawa S., et al., Nature. 2003 Apr. 10; 422(6932):583-8.
Yu et al. 2003. Nat. Neurosci.; 6 (11):1169-1177. (ref 10)
Zechner et al. 2003. Dev Biol; 258 (2):406-418. (ref 35)
Zetterstrom et al. Science 1997 Apr. 11; 276(5310):248-50
Zhang et al., 2001, Nat Biotech., 19, 1129-1133.
Zheng et al. 1996. Oncogene.; 12 (3): 555-562. (ref 25)

1. A method of inducing or promoting dopaminergic neuronal development by enhancing proliferation, self-renewal, dopaminergic induction, survival, differentiation and/or maturation in neural stem, progenitor or precursor cell, or other stem or neural cell, the method comprising:
expressing a nuclear receptor of the Nurr1 subfamily above basal levels within the cell, and
regulating GSK-3β inhibition within the cell other than by means of a ligand for a Frizzled receptor, thereby producing or enhancing proliferation, self-renewal, survival and/or dopaminergic induction, differentiation, survival or acquisition of a neuronal dopaminergic phenotype.

2. A method according to claim 1 wherein the nuclear receptor is Nurr1.

3. A method according to claim 1 wherein the nuclear receptor is Nor1 or NGFI-B.

4. A method according to claim 1 comprising expressing Nurr1 above basal levels by transforming a cell with Nurr1 DNA or introducing into the cell Nurr1 RNA.

5. A method according to claim 1 comprising expressing Nurr1 above basal levels by introducing Nurr1 protein into the cell.

6. A method according to claim 1 comprising expressing Nurr1 above basal levels by preserving Nurr1 protein in the cell.

7. A method according to claim 1 wherein GSK-3β inhibition is by means of a GSK-3β inhibitor selected from the group consisting of hymenaldisines, aloisines, maleimides, thiazoles, thienyl and methyl halomethyl ketones, oxidized aminopyrimidines; indirubines and paullones.

8. A method according claim 7 wherein the inhibitor is an oxidized aminopyrimidine or paullone.

9. A method according to claim 8 wherein the inhibitor is kenpaullone.

10. A method according to claim 1 wherein GSK-3β inhibition is by means of β-catenin stabilisation.

11. A method according to claim 1 wherein the neural stem, progenitor or precursor cell or other stem or neural cell is mitotic and/or capable of self-renewal when it is treated with the GSK-3β inhibition.

12. A method according to claim 1 wherein said neural stem, progenitor or precursor cell or other stem or neural cell is additionally contacted with a member of the FGF family of growth factors.

13. A method according to claim 1 wherein said neural stem, progenitor or precursor cell or other stem or neural cell is contacted with a retinoid or retinoid derivative, an activator of the retinoid X receptor (RXR), a repressor of the retinoid acid receptor (RAR), 9-cis retinal, DHA, SR11237, or LG849.

14. A method according to claim 1 wherein the neural stem, progenitor or precursor cell or other stem or neural cell is treated with bFGF and/or EGF and/or FGF-8 and/or LIF and/or Shh prior to or simultaneously with treating the cell with the regulation of GSK-3β inhibition.

15. A method according to claim 1 wherein the neural stem, progenitor or precursor cell or other stem or neural cell is grown in the presence of antioxidants, ascorbic acid, low oxygen tension or a hypoxia-induced factor.

16. A method according to claim 1 wherein the neural stem, progenitor or precursor cell or other stem or neural cell grows and/or differentiates in the presence of ventral mesencephalic astrocytes or early glial cells.

17. A method according to claim 1 wherein the regulation of GSK-3β inhibition is provided to an in vitro culture containing the cell.

18. A method according to claim 17 wherein the regulation of GSK-3β inhibition is produced by expression of a protein in the neural stem, progenitor or precursor cell, or other stem or neural cell, or by transformation with nucleic acid encoding a substance that provides the regulation of GSK-3β inhibition.

19. A method according to claim 17 wherein the regulation of GSK-3β inhibition is produced by expression from a cell co-cultured with the neural stem, progenitor or precursor cell, or other stem or neural cell, which co-cultured cell is a cell other than a type 1 astrocyte or early glial cell or is a host cell transformed with nucleic acid encoding a substance that provides GSK-3β inhibition or a cell containing introduced GSK-3β.

20. A method according to claim 19 wherein the co-cultured cell other than a type 1 astrocyte or early glial cell or host cell is another stem, neural stem, progenitor, precursor or neural cell.

21. A method according to claim 17 wherein the neural stem, progenitor or precursor cell, or other stem or neural cell, is engineered to express a substance that provides regulation of GSK-3β inhibition from encoding nucleic acid.

22. A method according to claim 17, wherein a substance providing regulation of GSK-3β inhibition is introduced into the cell.

23. A method according to claim 1 comprising further co-culturing the neural stem, progenitor or precursor cell, or other stem or neural cell, with an early glial cell, or a Type 1 astrocyte optionally of the ventral mesencephalon.

24. A method according to claim 23 wherein the Type 1 astrocyte is immortalized or is of an astrocyte cell line of a region other than the ventral mesencephalon.

25. A method according to claim 1, comprising additionally contacting the neural stem, progenitor or precursor cell, or other stem or neural cell with a negative selection agent that selects against non-dopaminergic neurons.

26. A method according to claim 1 further comprising formulating a neuron into a composition comprising one or more additional components.

27. A method according to claim 26 wherein the composition comprises a pharmaceutically acceptable excipient.

28. A method according to claim 27 further comprising administering the composition to an individual.

29. A method according to claim 28 wherein the neuron is implanted into the brain of the individual.

30. A method according to claim 1 wherein the cell is treated in an individual in situ to increase Nurr1 expression.

31. A method according to claim 1 wherein the neural stem, progenitor or precursor cell or other stem or neural cell is treated in an individual in situ with a substance regulating GSK-3β inhibition, other than a ligand for a Frizzled receptor.

32. A method according to claim 31, wherein nucleic acid encoding the substance regulating GSK-3β inhibition is introduced into the cell.

33. A method according to claim 31, wherein the substance regulating GSK-3β inhibition is introduced into the cell.

34. A method according to claim 31 wherein the cell is treated in an individual in situ to increase Nurr1 expression above basal levels.

35. A method according to claim 30 wherein the neural stem, progenitor or precursor cell or other stem or neural cell is endogenous to the individual.

36. A method according to claim 30 wherein the neural stem, progenitor or precursor cell or other stem or neural cell is exogenously supplied by grafting into the individual.

37. A method according to claim 28 wherein the individual has Parkinson's disease, a parkinsonian syndrome, neuronal loss or a neurodegenerative disease.

38-42. (canceled)

43. A method of obtaining a factor or factors which, either alone or in combination, enhance proliferation, self-renewal, survival and/or dopaminergic development, induction, differentiation, or maturation in a neural stem, progenitor or precursor cell, or other stem or neural cell expressing Nurr1 above basal levels, the method comprising:
(a) treating a neural stem progenitor or precursor cell, or other stem or neural cell expressing Nurr1 above basal levels with a regulator of GSK-3β inhibition in the presence and absence of one or more test substances; and
(b) determining proliferation, self-renewal, survival and/or dopaminergic development, induction, differentiation, or maturation of the cell and comparing the extent of the proliferation, self-renewal, survival and/or dopaminergic development, induction, differentiation or maturation in the presence and absence of the test substance or substances, whereby said factor or factors is obtained.

44. A method according to claim 43 wherein the cell is treated with a regulator of GSK-3β inhibition in in vitro culture containing the cell.

45. A method according to claim 43, wherein the cell is treated with a regulator of GSK-3β inhibition by introduction of nucleic acid encoding the regulator into the cell.

46. A method according to claim 43, wherein the cell is treated with a regulator of GSK-3β inhibition by introduction of the regulator into the cell.

47. A method according to claim 43 further comprising co-culturing the neural stem, progenitor or precursor cell, or other stem or neural cell with an early glial cell or a Type 1 astrocyte optionally of the ventral mesencephalon.

48. A method according to claim 43 wherein a factor or factors able to enhance proliferation, self-renewal, survival and/or dopaminergic development, induction, differentiation or maturation in a neural stem, progenitor or precursor cell, or other stem or neural cell expressing Nurr1 above basal levels is or are provided in isolated and/or purified form.

49. A method according to claim 43 wherein a factor or factors able to enhance proliferation, self-renewal, survival and/or dopaminergic development, induction, differentiation or maturation in a neural stem, progenitor or precursor cell, or other stem or neural cell expressing Nurr1 above basal levels is or are formulated into a composition comprising one or more additional components.

50. A method according to claim 49 wherein the composition comprises a neural stem, progenitor or precursor cell, or other stem or neural cell expressing Nurr1 above basal levels.

51. A method according to claim 49 wherein the composition comprises a pharmaceutically acceptable excipient.

52. A method according to claim 51 further comprising administering the composition to an individual.

53. A method according to claim 52 wherein the composition is implanted into the brain of the individual.

54. A method according to claim 53 wherein the individual has Parkinson's disease, a parkinsonian syndrome, neuronal loss or a neurodegenerative disease.

55-60. (canceled)

61. A method of obtaining an agent that induces or promotes dopaminergic neuronal development, the method comprising determining ability of a test substance to regulate inhibition of GSK-3β in a neural stem, progenitor or precursor cell, or other stem or neural cell, wherein the cell expresses a nuclear receptor of the Nurr1 subfamily above basal levels within the cell, or a dopaminergic neuron.

62. A method according to claim 61 comprising determining ability of a test substance to provide GSK-3β inhibition in the cell.

63. A method according to claim 61 or comprising determining ability of a test substance to inhibit GSK-3β in the cell.

64. A method according to claim 61 comprising determining ability of a substance to stabilise β-catenin in the cell.

65. A method of obtaining an agent that induces or promotes dopaminergic neuronal development, the method comprising
(i) determining ability of a test substance to regulate inhibition of GSK-3β, thereby identifying a substance that regulates GSK-3β inhibition,
(ii) determining ability of a substance that regulates GSK-3β inhibition identified in (i) to induce or promote dopaminergic neuronal development by providing a neural stem, progenitor or precursor cell, or other stem or neural cell, wherein the cell expresses a nuclear receptor of the Nurr1 subfamily above basal levels within the cell, with the substance that regulates GSK-3β inhibition, thereby obtaining said agent.

66. A method according to claim 65 wherein the substance that regulates GSK-3β inhibition inhibits GSK-3β.

67. A method according to claim 65 wherein the substance that regulates GSK-3β inhibition stabilises β-catenin.

68. A method according to claim 61 further comprising formulating the agent into a composition comprising one or more additional components.

69. A method according to claim 68 wherein the composition comprises a neural stem, progenitor or precursor cell, or other stem or neural cell expressing Nurr1 above basal levels.

70. A method according to claim 68 wherein the composition comprises a pharmaceutically acceptable excipient.

71. A method according to claim 70 further comprising administering the composition to an individual.

72. A method according to claim 71 wherein the composition is implanted into the brain of the individual.

73. A method according to claim 72 wherein the individual has Parkinson's disease, a parkinsonian syndrome, neuronal loss or a neurodegenerative disease.

74. A method of detection of presymptomatic Parkinson's disease or diagnosis or confirmation of diagnosis of Parkinson's disease, the method comprising determining in a patient or a sample removed from a patient the presence of an abnormality in GSK-3β inhibition.

75. A method according to claim 74 wherein the presence of an abnormality in GSK-3β inhibition is determined in neurons removed from the patient.

76. A method according to claim 74 wherein abnormality is determined in GSK-3β activity.

77. A method according to claim 74 wherein abnormality is determined in β-catenin stabilisation.

78. A method according to claim 31, wherein the neural stem, progenitor or precursor cell or other stem or neural cell is endogenous to the individual.

79. A method according to claim 31, wherein the neural stem, progenitor or precursor cell or other stem or neural cell is exogenously applied by grafting into the individual.

80. A method according to claim 65 further comprising formulating the agent into a composition comprising one or more additional components.
